(19) United States
(12) Patent Application Publication
(10) Pub. No.: US 2021/0179940 A1
(43) Pub. Date: Jun. 17, 2021

(54) WIDE TEMPERATURE RANGE AND HIGH PHOTOCHEMICAL STABILITY SMECTIC LIQUID CRYSTAL COMPOSITIONS WITH A MONOLAYER SMECTIC A PHASE (SMA1), METHOD OF OBTAINING THEREOF AND DEVICES UTILIZING THEREOF

(71) Applicant: WOJSKOWA AKADEMIA TECHNICZNA, Warszawa (PL)

(72) Inventors: Roman DABROWSKI, Warszawa (PL); Wiktor PIECEK, Zaborow (PL); Magdalena URBANSKA, Warszawa (PL); Jerzy DZIADUSZEK, Warszawa (PL); Dorota WEGLOWSKA, Zaborow (PL); Rafal MAZUR, Piszczac (PL); Jakub BOZETKA, Rybnik (PL)

(73) Assignee: WOJSKOWA AKADEMIA TECHNICZNA, Warszawa (PL)

(21) Appl. No.: 16/760,228

(22) PCT Filed: Oct. 10, 2018
(86) PCT No.: PCT/EP2018/077651

§ 371 Date: Apr. 29, 2020

(30) Foreign Application Priority Data
Oct. 31, 2017 (PL) P.423327

(51) Int. Cl. C09K 19/12; C09K 19/34; C09K 19/58; C09K 19/02

(52) U.S. Cl. CPC C09K 19/12; C09K 19/3447; C09K 19/584; C09K 19/0225; C09K 2019/3016; C09K 19/3458; C09K 2019/122; C09K 2019/123; C09K 2019/124; C09K 19/0266

(57) ABSTRACT

A smectic A composition with the positive dielectric anisotropy exhibiting a monolayer smectic A structure (SmA₁), which shows a phase transition from a smectic A phase to an isotropic phase (SmA-Iso) or a phase transition from a smectic A phase through a nematic phase to an isotropic phase (SmA-N-Iso) and includes at least two fluorinated compounds selected from the families of fluorinated derivatives of biphenyls and terphenyls expressed by the general formulae 1-12.

For ensuring of the high level of the conductivity the composition is doped with the ionic amidynium salts and/or the ionic potassium complexes of the crown ethers wherein the charge on the cation is delocalized. Such a structure of the ionic compound ensures the better solubility in the liquid crystalline fluorinated composition in comparison to that observed for quaternary ammonium salts.

For writing of the information the dynamic scattering effect (DS) was used, where the transition from a opaque (milky) state to a clear (transparent) state or reversal process is obtained by the change of the frequency of a driving alternating electric field.

The modification of the properties of the smectic A composition by adding of further components such as fluorinated four ring liquid crystal compounds or pyrimidine compounds or cyanocompounds is described.

### TECHNICAL FIELD

This application refers to Polish Patent application P.423327, filed on 31 Oct. 2017.

The aspects of the disclosed embodiments relate to liquid crystal smectic compositions with positive dielectric anisotropy and high photochemical stability exhibiting a monolayer smectic A phase (SmA₁), which after adding ionic dopants are used to manufacturing smart windows and memory displays, wherein a scattering state (milky) and a clear state is obtained by the change of a frequency of an electric field, respectively.

### BACKGROUND

In the patents or papers for the description of liquid crystal compositions alternatively terms are used: liquid crystal medium or liquid crystal mixture or liquid crystal material as meaning the same.

Nematic liquid crystalline compositions are commonly used to present information in the different kinds of digital and literal displays or large flat screens addressed by active matrix wherein different electrooptic effects are used, such as TN, STN, ECB, VAN or IPS. Many chemically stable nematic liquid crystalline compositions with short response times were elaborated for these purposes, and they are well known now at the state of the art and produced by different commercial firms, see Refs. as examples: J. A. Castellano, Handbook of Display Technology, Academic Press, Inc. San Diego, New York (pp. 181-251); M. Hird Fluorinated liquid Crystals—properties and applications, Chem. Soc. Rev., 36, pp. 2070-2095 (2007); T. Geelhaar, Liquid crystals for displays applications, Liq. Cryst., 24, pp. 91-98 (1998); Patent PL 217 692 B1; liquid crystals catalogues of Merck, Chisso and Dainippon Ink.

Smectic liquid crystalline compositions, except of those with ferro- or antiferroelectric properties, characterize by slow response on the change of an electric field and therefore their use is limited, but they become more important at applications, wherein information is not changed quickly or information is presented for a long time—e.g. information notices, electronic books or smart windows. Using a smectic A composition (SmA) a long time of life of recorded information may be obtained without a need of application of a refreshing voltage. The addressing with voltage application is necessary only when information is written or erased.

For this purpose a dynamic scattering effect (DS) and a liquid crystalline material doped with ionic compounds are used.

D. Coates and co-workers (J. Phys. D: Appl. Phys., II, 20215-34 (1978)) demonstrated such display at first time. The other examples of similar displays were presented by i.e.

N. V. Chirkov and co-workers: N. V. Chirkov, D. F. Aliev, Mol. Cryst. Liq. Cryst., 49, 293-8 (1979), D. Coates and co-workers, Eurodisplay 1987 (London), 96-99 (1987); D. F. Aliev and co-workers. Mol. Cryst. Liq. Cryst., 213, 137-43 (1992); H. Coates and co-workers. Proc. SPIE, 2408, 14-21 (1995); D. J. Gardiner and co-workers, Proc. SPIE, 5741, 239-47 (2005); D. J. Gardiner and co-workers, J. Appl. Phys., 99, 3577 (2006); D. J. Gordiner and co-workers., J. Phys. D, Appl. Phys., 39, 4948-55 (2006); W. Ji and co-workers, Opt. Mat. Express, 5, 280-286 (2015).

In the DS effect two stable states are observed: the first one is clear (transparent) and the second one is opaque (milky, scattering). They are a result of different mobility of ions under an influence of a driving electric field of various frequencies.

Ions movement in the alternating electric field at a low frequency involves a turbulent flow of domains formed from smectic A layers with homeotropically oriented molecules—then a confocal, strongly scattering texture is formed which is preserved also when an electric field is switched off. If there is a liquid crystalline material placed between the substrates with a rough surface the observed threshold voltage of the scattering state is lower than for that with a more smooth surface.

For an electric field of a higher frequency (higher than 1 kHz) the long distance movement of ions is hindered and the electric field together with a surface agent orient the molecular director in a homeotropic way (perpendicular) to the electrode surfaces—a clear (transparent) state is observed now. This state is preserved after an electric field is switched off.

A smectic liquid crystalline composition for dynamic scattering effect (DS) ought to characterize by a positive dielectric anisotropy (ε_{II}−ε_{⊥}=Δε>0) and a negative anisotropy of conductivity (δ_{∥}−δ_{⊥}<0).

The higher absolute values of parameters mentioned above are the lower threshold voltage is (voltage needed to start a turbulent movement—to write information in scattering state). Only a few kinds of liquid crystal smectic A materials were considered as convenient for the induction of the DS effect. In the most cases they were compositions formulated with cyanocompounds. In U.S. Pat. No. 4,139,272 the composition composed of alkylcyanobiphenyls and alcoxycyanobiphenyls was described.

It is the smectic A composition which characterizes by partially bilayer structure (SmA_{d}), wherein ratio d/L˜1.4, d—the layer spacing (thickness), L—the molecule length. The composition was doped with hexadecyltrimethyl ammonium bromide [C₁₆H₃₃N(CH₃)₃]⁺Br⁻ in amount of 0.1 wt % in purpose to obtain desired homeotropic orientation and the desired level of conductivity. The same kind of the liquid crystalline composition was used for manufacturing displays addressed with an electric field in U.S. Pat. Nos. 4,419,664 and 4,528,562.

In U.S. Pat. No. 4,291,948 a smectic liquid crystalline material was used to obtain a color information display, which consists of 4-cyano-4-octylbiphenyl or 4-pentylphenyl 2-chloro-4-(6-hexylnaphtalenecarbonyloxy-2)benzoate or 4-octylphenyl 4-trans-butylcyclohexane-1-carboxylate doped with an antraquinone dye and hexadecyltrimethyl ammonium bromide.

In U.S. Pat. No. 4,834,904 a multicomponent composition composed of alkylcyanobiphenyls, alcoxycyanobiphenyls, alkylcyanoterphenyls and comprising additionally other three ring compounds such as alkylcyclohexylcyanobiphenyls, al kylphenylethylcyanobiphenyls and cyanobiphenyl al kylcyclohexanocarboxylates was described. It had the smectic A phase of A_{d} type in the broad temperature range (−17° C.-65° C.). In US Patent applications 2013/0155340A1 and 2013/0342773A1 the smectic A composition comprising a liquid crystalline compound with a terminal oligosiloxanyl group being the analogue of an alcoxycyanobiphenyl and a siloxane polymer doped an ammonium salt-preferably ammonium chlorate (VII) of formula [C₁₃H₂₇N]CH₃)₃]⁺ClO₄⁻ was described.

In US Patent application 2012/0032994 A1 an induced smectic A composition comprising siloxane compounds and a commercial nematic mixture was described.

The composition composed of alcoxycyanobiphenyl and alkoxycyanobiphenyls with the terminal chain modified by joining a siloxane group (CH₃)₃SiOSi(CH₃)₂—CₙH₂ₙO was also described by D. J. Gardiner et al. (J. Phys. D; Appl. Phys. 39, 4948-4955 (2006), a similar smectic composition and also a smectic composition comprising terphenyl, biphenyl and phenyl pyridine isothiocyanates were investigated by W. Ji et al., Optical Material Express, 5, 281-286 (2015).

In U.S. Pat. No. 7,678,292B2 and equivalent EP Patent 1537191B1 different quaternary ammonium salts as ionic dopants for the modulation of the dynamic scattering effect were claimed. The composition comprising cyanobiphenyls was used here.

The best ionic dopants were ionic compounds: hexadecyltrimethylammonium 4-nonyloxybenzoate or 4-dodecylbenzenesulphonate or 4-nonyloxybenzenephosphonate.

It is known also another method to write information on the smectic A, wherein a laser emitting infrared radiation (IR) is used. A smectic A material is heated by laser rays to above an isotropic point and then it is cooled rapidly without an electric field. A strongly scattering state is observed. When the smectic A material is cooled slowly from the isotropic state the transparent structure (homeotropic one) is observed, where the electrode surfaces are covered with siloxane orienting agent (see Ref. F. J. Kahn, Appl. Phys. Lett., 22, 111-113 (1973)). Schiff's base was used here.

The dynamic scattering effect need not polarizers. It decreases the cost of devices from one side, but from the other side a destructive action of ultraviolet radiation is easier.

Therefore the liquid crystalline compositions with a high photostability are necessary to be used in applications. Desired features possess fluorinated liquid crystals. Such compounds are commonly used in nematic compositions (see for example T. Geelhear, Liq. Cryst., 24, 91-98 (1988), D. Pauluth et al., J. Mater. Chem., 14, 1219-27 (2004)).

Only liquid crystal structure compositions with a broad temperature range of tilted smectic C phase composed of fluorinated compounds are known, see for example J. W. Goodby et al., Proc. of SPIE, 3955, 2-34 (2000).

Smectic A compositions obtained from fluorinated compounds have not described or used yet, although numerous single compounds with this phase are known.

Fluorinated compounds are characterized by a high electric resistivity, what is the results of a small ability to solvation of ions and their small extraction from electrodes. In the case of nematic compositions it is their important and preferable feature, because it enables obtaining of compositions with a high resistivity which is necessary to address displays by an thin layer active matrix.

The same feature is not preferable in the case of smectic compositions, because a desired level of conductivity in the range of 10⁻⁹ Ohm⁻¹cm⁻¹ or higher is difficult to obtain.

Fluorinated derivatives of biphenyl and terphenyl, especially those substituted in the lateral position in a vicinal way, are commonly used as the components of the nematic compositions for the VAN effect. They are also important components of mixtures with the smectic C phase, e.g. ferroelectric compositions (see W. Grey et al., J. Chem. Soc. Perkin Trans. II, 2041-2053 (1989), J. W. Goodby et al., Proc of SPIE, 3955, 2-34 (2000)).

Compositions which are characterized with a broad temperature range of the smectic A phase consisted of fluorinated derivatives of biphenyls and terphenyls are not known yet.

### Disclosure of the Embodiments—as in Prev Ver

According to the aspects of the disclosed embodiments a smectic A composition with the positive dielectric anisotropy exhibiting a monolayer smectic A structure (SmA₁), which shows a phase transition from a smectic A phase to an isotropic phase (SmA-Iso) or a phase transition from a smectic A phase through a nematic phase to an isotropic phase (SmA-N-Iso) and comprises at least two fluorinated compounds selected from the families of fluorinated derivatives of biphenyls and terphenyls expressed by the general formulae 1-12,

wherein the terminal chain R1 is an alkyl group (H₂ₙ₊₁Cₙ) containing from 1 to 15 carbon atoms (n=1-15), the terminal chain R2 is an alkyl group (CmH₂ₘ₊₁) or an alcoxy group (OCₘH₂ₘ₊₁) or an alkyl carbonato group (OCOOCₘH₂ₘ₊₁), each containing independently from 1 to 15 carbon atoms (m=1-15); and wherein at least one of the said fluorinated compounds is a compound expressed by the general formulae 1 or 2 or 3;

Preferably, the composition contains additionally
at least one four-ring fluorinated compound preferably selected from the compounds expressed by the general formulae 13-18:
or at least one derivative of pyrimidine expressed by the general formulae 19-20
or a derivative of terphenyl of the general formula 21
or two or all said compounds,
   wherein terminal group R1 is an alkyl group (H₂ₙ₊₁Cₙ) containing from 1 to 15 carbon atoms (n=1-15), R3 and R4 are independently an alkyl or an alcoxy or an alkyl carbonato group, each containing from 1 to 15 carbon atoms.

Preferably, the composition contains additionally at least one liquid crystalline cyanocompound preferably selected from compounds expressed by the general formulae 4-12, wherein the terminal fluorine atom (F) is replaced by the cyano group (CN) and its concentration is below 25 wt. %, preferably in range of 3 wt. % to 20 wt. %.

Preferably, the composition contains additionally at least one or more ionic dopants with delocalized charge of cation selected from ionic compounds expressed by the general formulae 22-26, wherein the substituents R10, R11, R13 and R15 in the cationic part of the molecule are independently a hydrogen atom or an alkyl group or an alkylcyclohexyl group or a phenyl group or an alkylphenyl group and they are the same or different and contain 1 to 25 carbon atoms; the substituents R12 and R14 are an alkyl group containing independently from 1 to 25 carbon atoms, preferably from 12 to 18 carbon atoms; in the formula 23 symbols A and B denote a part of the ring containing from two to five methylene groups leading to specially preferable compounds with the system of rings expressed by formulae 24 (A=5, B=3) or 25 (A=4, B=2), wherein R14 is preferably an alkyl group containing from 2 to 18 carbon atoms; the anion Y⁻ denotes an organic or an inorganic anion preferably: Cl^{θ}— (a), Br^{θ}— (b), ClO₄^{θ}— (c), CF₃SO₃^{θ}— (d), R5-SO₃^{θ}— (e), SCN^{θ}— (f),

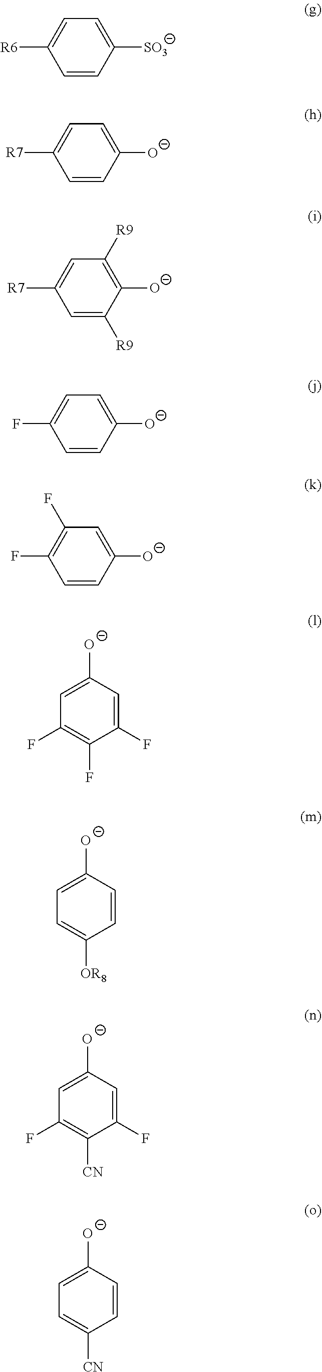

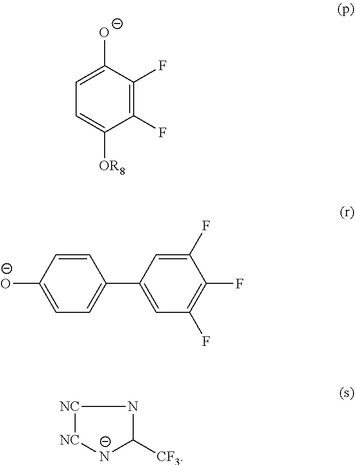

wherein substituents R5, R6, R7 and R8 are meaning a hydrogen or an normal alkyl group or an iso-alkyl group or a sec-alkyl group each containing independently from 1 to 20 carbon atoms and R9 is a branched alkyl group preferably the tert-butyl group.

Preferably, the composition contains additionally at least one or more ionic dopants with a delocalized charge of cation selected from ionic compounds being complexes formed from the salts of alkaline metals preferably from potassium salts and crown ethers preferably expressed by the general formulae 27 and 28, wherein symbol A is meaning a cyclohexane ring or a benzene ring, substituents R16 are independently a hydrogen atom or an alkyl group the same or different containing each from 1 to 16 carbon atoms; anion Y⁻ is an organic anion or an inorganic anion preferably: Cl^{θ}— (a), Br^{θ}— (b), ClO₄^{θ}— (c), CF₃SO₃^{θ}— (d), R5-SO₃^{θ}— (e), SCN^{θ} (f)

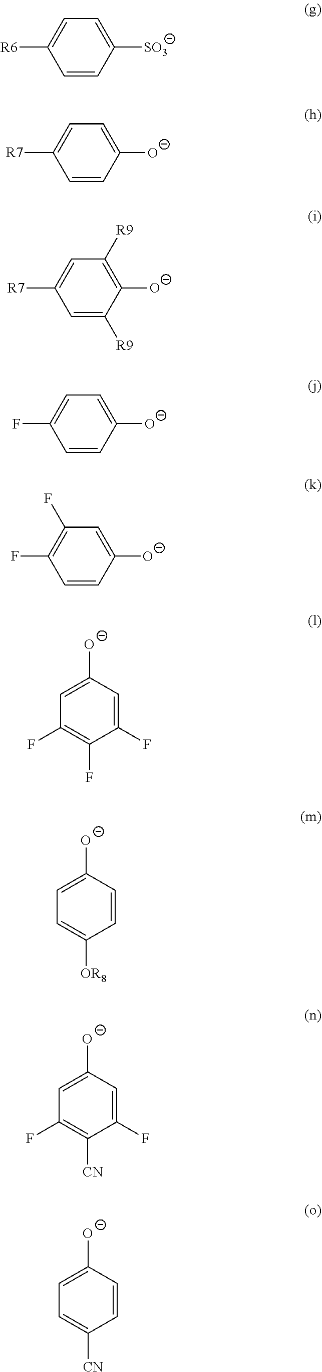

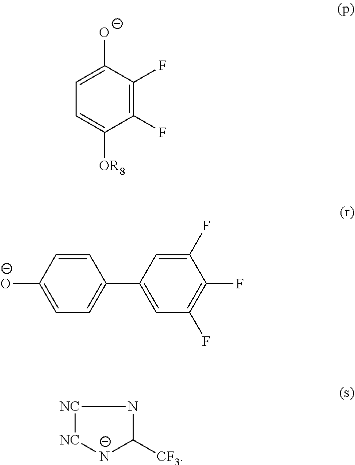

R5, R6, R7 and R8 are meaning a hydrogen or an normal alkyl group or an iso-alkyl group or a sec-alkyl group each containing independently from 1 to 20 carbon atoms and R9 is a branched alkyl group preferably the tert-butyl group

Preferably, the composition contains additionally simultaneously two kinds of ionic dopants—as defined in the two preceding paragraphs.

Preferably, to such composition a dye or a few dyes is/are added with isotropic or anisotropic absorption of light with preferably of formula 34 or 35 or an nonionic structure preferable an anthraquinone dye in the amount below of 3 wt. %, preferably in range 0.5 wt % to 1.5 wt. %.

According to the aspects of the disclosed embodiments an ionic compound expressed by the general formulae 22-26; wherein substituents R10, R11, R13 and R15 in cationic part of the molecule are meaning independently a hydrogen atom or an alkyl group or an alkyl cyclohexyl group or a phenyl group or an alkyl phenyl group and they are the same or different and contain 1 to 25 carbon atoms; the substituents R12 and R14 are an alkyl group containing independently from 1 to 25 carbon atoms, preferably from 12 to 18 carbon atoms; in the formula 23 symbols A and B denote a part of the ring containing from two to five methylene group leading to specially preferable compounds with the system of rings expressed by formulae 24 (A=5, B=3) or 25 (A=4, B=2), wherein R14 is preferably an alkyl group containing from 2 to 18 carbon atoms;

the anion Y⁻ denotes an organic or an inorganic anions preferably: Cl^{θ}— (a), Br^{θ}— (b), ClO₄^{θ}— (c), CF₃SO₃^{θ}— (d), R5-SO₃^{θ}— (e), SCN^{θ} (f)

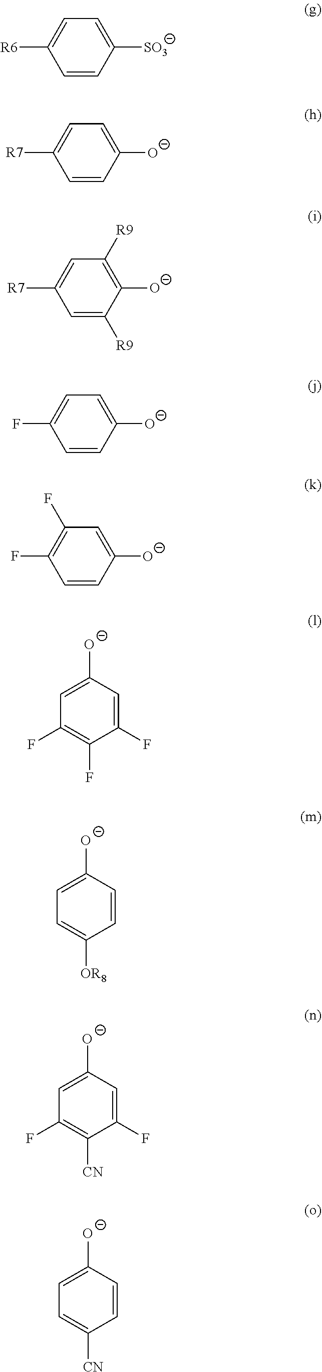

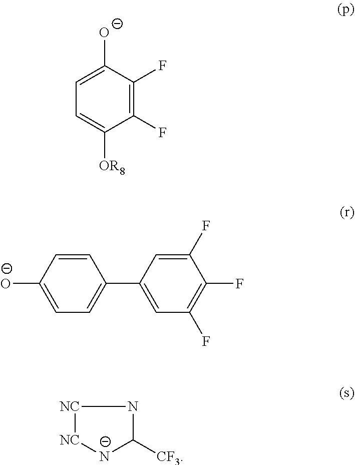

wherein substituents R5, R6, R7 and R8 denote a hydrogen or an normal alkyl group or an iso-alkyl group or a sec-alkyl group each containing independently from 1 to 20 carbon atoms, R9 is a branched alkyl group preferably the tert-butyl group used as an ionic dopant to increase conductivity in smectic A compositions.

According to the aspects of the disclosed embodiments an ionic compound expressed by the general formulae 27 and 28; wherein symbol A is a cyclohexane ring or a benzene ring, substituents R16 are independently a hydrogen atom or an alkyl group the same or different containing each from 1 to 16 carbon atoms; anion Y⁻ is an organic anion or an inorganic anion preferably: Cl^{θ}— (a), Br^{θ}— (b), ClO₄^{θ}— (c), CF₃SO₃^{θ}— (d), R5-SO₃^{θ}— (e), SCN^{θ} (f)

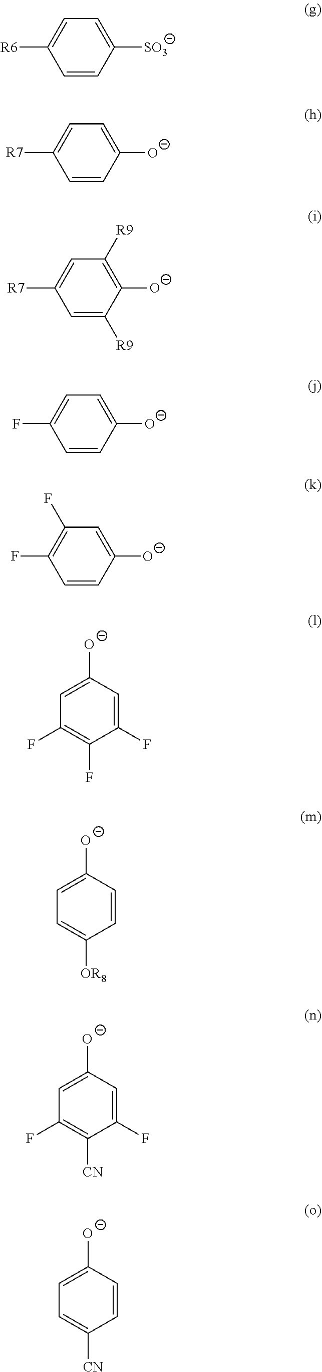

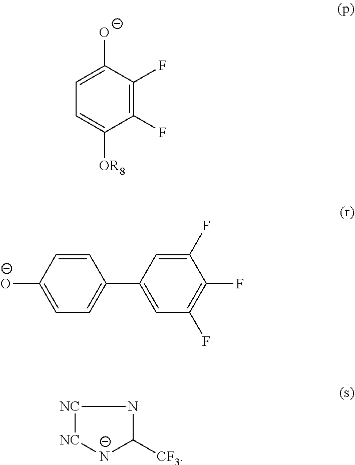

used as an ionic dopant to increase conductivity in smectic A compositions.

According to the aspects of the disclosed embodiments a method of obtaining of a smectic A composition doped with the above-mentioned ionic compound characterized in that the above-mentioned composition is dissolved in an organic solvent preferably in flurobenzene or chloroform and an ionic compound as defined in the two preceding paragraphs is dissolved in an organic solvent preferably fluorobenzene or chloroform, then both solutions are combined, then mixed, and filtered through micropore membrane; the solvent is evaporated at first under normal pressure then at lower pressure, the rest is heated under flow of an inert gas; the ionic dopant is added in such amount to obtain conductivity of order of 10⁻⁹ Ohm⁻¹ cm⁻¹ or higher.

Preferably, to the solution containing liquid crystal composition a compound is added additionally with ability to hinder radical reaction from a phenol family expressed by the formula 29, wherein substituent R9 in orto-position to hydroxy group is a bulk substituent preferably tert-butyl group or an amine derivative expressed by formulae 30-33, wherein the substituent R17 denotes preferably a hydrogen atom or a benzyl group or a benzoyl group.

According to the aspects of the disclosed embodiments a device being a smart window or a memory display which consists of a two plastic or glass substrates covered inside with conductive and/or orienting layer, wherein both electrodes are transparent or one contains a reflective layer from a metal preferably aluminum and the gap between substrates is in the range of 5-20 μm, preferably 15 μm, characterized in that said device is filled with a said liquid crystalline composition and prepared by the said method.

Preferably, the filling process is carried in an inert atmosphere, preferably in argon for said memory display or in crypton for said smart window and wherein the liquid crystal smectic A composition used is earlier carefully degassed and is stored in said inert atmosphere.

According to the aspects of the disclosed embodiments a pyrimidine derivative expressed by the general formulae 19 and 20; wherein a left terminal substituent R3 is an alkyl carbonato group (H₂ₙ₊₁Cₙ)OCOO and right terminal substituent R4 is an alkyl group CₘH₂ₘ₊₁ or an alcoxy group OCₘH₂ₘ₊₁ or left substituent R3 is an alkyl group H₂ₙ₊₁Cₙ or an alcoxy group H₂ₙ₊₁CₙO and right substituent R4 is alkyl carbonato group OCOO CₘH₂ₘ₊₁ in which symbols n and m denote independently numbers from 1 to 15.

### BRIEF DESCRIPTION OF THE DRAWINGS

Preferred embodiment of the present disclosure are presented in a more detailed way with reference to the attached drawing, in which:

FIG. 1 presents a hypothetic placement of liquid crystalline molecules (see the composition 18) in the single smectic A₁ layer.

FIG. 2 shows the dependence of light transmission (I) while the switching of erase state (clear)—black line and write state (scattering)—red line versus voltage for mixture 18 doped with ionic dopant II.16b with concentration 0.3 wt. %. Vₜₕ(sc)—threshold voltage, Vₛ(sc)—saturation voltage for the scattering state obtained at the frequency f_{sc}=50 Hz of the driving electric field. Vₜₕ(c)—threshold voltage, Vₛ(c)—saturation voltage for the clear state obtained at the frequency f_{c}=0.8 kHz of the driving electric field. The observation was done for the transmitted light (collimated beam of white light from a stabilized halogen source) intensity passing through a flat-parallel measuring cell with cell gap of c.a. 15 μm. The light intensity was observed with linear photo detector placed 12 cm behind the cell.

### DETAILED DESCRIPTION

Preferred embodiments of the present disclosure are described in details below. The examples serve only as an illustration and do not limit the scope of the present disclosure.

The presented disclosure includes several new aspects: the first one refers to a new photostable liquid crystal composition, the second one refers to a ionic compounds with a good solubility in the fluorinated biphenyl and terphenyl derivatives composition, and third one refer to a device filled with said liquid crystalline composition comprising said ionic dopant.

It was unexpectedly discovered that liquid crystalline smectic A composition with a broad temperature range of this phase may be obtained from fluorinated derivatives of biphenyl or terphenyl, wherein a direct transition from the smectic A phase to the isotropic phase is observed (SmA-Iso) or an indirect transition from the smectic A phase through the nematic phase to the isotropic phase is observed (SmA-N-Iso).

The presence of nematic phase before the isotropization point (clearing point) enables to obtain better homeotropic texture during filling of a cell.

It was found that the composition with the smectic A phase may be obtained from the same chemical families, which are used to manufacturing a nematic composition or a composition with the smectic C phase if said compounds and their concentration are selected in a proper way.

The first aspect of the disclosed embodiments is a smectic A composition with a positive dielectric anisotropy exhibiting monolayer smectic A structure (smectic A₁). Here the average layer spacing (thickness) is nearly equal the average length of molecules (R. Dqbrowski, Liq. Cryst., 42, 738-818, 2015).

The composition according to the aspects of the disclosed embodiments has the SmA₁-Iso phase transition or SmA₁-N-Iso phase transition and comprises at the least two fluorinated compounds selected from the families of fluorinated derivates of biphenyls and terphenyls expresses by the general formulae 1-12:

| | Acronym |
|---|---|
| 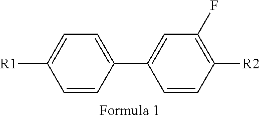 | 1n-m, 1n-Om, 1n-OCOOm |
| 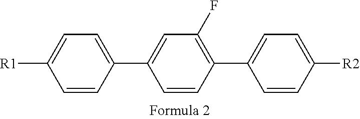 | 2n-m, 2n-Om, 2n-OCOOm |
| 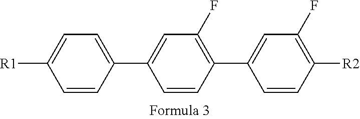 | 3n-m, 3n-Om, 3n-OCOOm |
| 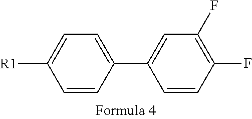 | 4n |
| 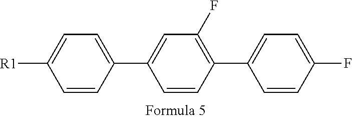 | 5n |
| 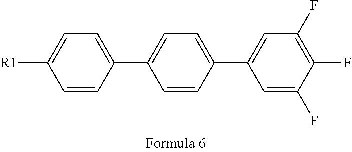 | 6n |
| 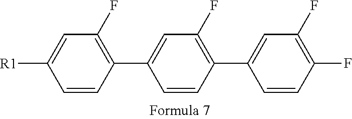 | 7n |
| 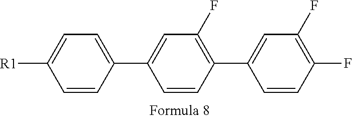 | 8n |
| 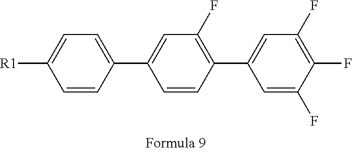 | 9n |
| 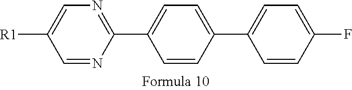 | 10n |
| 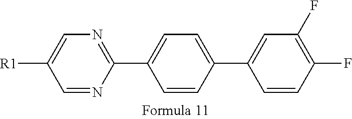 | 11n |
| 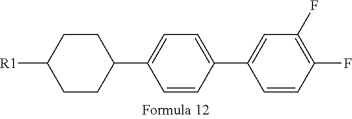 | 12n |

and wherein at least one fluorinated compound is a compound expressed by the general formulae 1 or 2 or 3.

In compounds 1-3 the terminal chain R1 is an alkyl group containing from 1 to 15 carbon atoms (H₂ₙ₊₁Cₙ—in short n), the terminal chain R2 is an alkyl group (H₂ₘ₊₁Cₘ—in short m) or an alcoxy group (OCₘH₂ₘ₊₁—in short Om) or an alkylcarbonato group (OCOOCₘH₂ₘ₊₁—in short OCOOm), each containing independently from 1 to 15 carbon atoms in CₘH₂ₘ₊₁ unit. The compounds 10, 11, 12 may be also considered as biphenyl derivatives in which terminal group is an alkylpyrimidynyl group or an alkylcyclohexyl group, respectively.

The compounds 1-3 have acronyms (notation): 1n-m, 1n-Om, 1n-OCOOm, 2n, 2n-Om, 2n-OCOOm, 3n-m, 3n-Om, 3n-OCOOm. The compounds 4-12 have acronyms: 4n, 5n, 6n, 7n, 8n, 9n, 10n, 11n, 12n.

The increase of concentration of compounds 3-12 in the composition according to the aspects of the disclosed embodiments involves the increase of its positive dielectric anisotropy and simultaneously decrease of threshold and saturation voltages necessary to obtain a clear state from a scattering state in a device which using the composition. The compounds 3-12 with short alkyl chains are favoring the presence of the nematic phase above the smectic A₁ phase (SmA₁-N-Iso phase sequence is obtained).

The observed temperature of the phase transition SmA₁-N in the obtained composition is higher than the calculated one from the properties of pure components. A smectogenic nature of the prepared composition is strengthened in the comparison to smectic nature of its components, when the compounds 1-3 are mixed with the compounds 4-12.

The examples of advantageous compounds 1-12 as components for obtaining the composition according to the present disclosure are listed in Table 1. The compounds exhibiting the enantiotropic smectic and nematic phases as well as compounds having only the nematic phase as well as compounds without a presence of these phases above melting points (the compounds with a monotropic or virtual nematic and smectic phases) are also preferable.

A characteristic feature of all compounds 4-12 is such that their parallel components of dipole moments are pending in the same direction and their adding leads to the increase of the total dielectric anisotropy and follows to the lower values of electric field needed for the reorientation of molecular director.

The smectic A phase observed in the composition according to the present disclosure has a monolayer structure (layer spacing d is near equal the average length of molecules (d˜Lₐᵥ), while mixtures composed with cyanobiphenyls used before have a partially bilayer structure (smectic A_{d} d>L, d˜1.4 L). The smectic A_{d} layers are composed of a mixture of dimeric and monomeric molecules (see R. Dqbrowski, Liq. Cryst., 42, 783-818 (2017)).

The smectic A₁ layers in comparison to the smectic A_{d} layers are more rigid and have less diffusive nature.

To increase a diffusive nature of the smectic A₁ layers it is proposed to add to the said composition a derivative of pyrimidine or terphenyl, which shows the phase sequence SmC-SmA-N-Iso or SmC-N-Iso or SmC-Iso. Molecular director in the smectic C layers is tilted to the normal to smectic layers, while in the smectic A layers is parallel to the normal to the smectic layers planes. Further difference is such that molecules in the smectic A layer rotate near freely, while the rotations of molecules in the smectic C layers are more hindered.

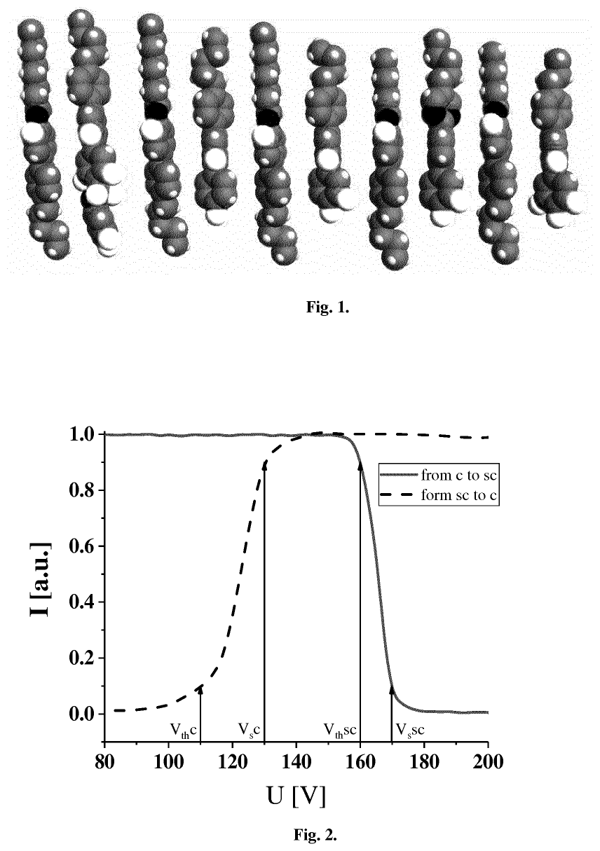

A statistic orthogonal order in the smectic A₁ phase is disturbed in the presence of pyrimidine molecules and its disordered character is increased. The number of defects in the smectic A layer is increased and the movement of ions in the direction perpendicular to electrodes becomes easier. A further increasing a diffusive nature of smectic layers involves the presence of four ring molecules, see FIG. 1.

This aspect of the present disclosure is described in the following way: the smectic A composition according the present disclosure modified in such way that it contains additionally at least one four-ring fluorinated compound preferably selected from the compounds expressed by the general formulae 13-18 or at least one a derivative of a pyrimidine expressed by the general formulae 19-20 or derivative of terphenyl 21 or two or all said compounds, wherein the terminal chain R1 has the same meaning as in the compounds 1-12, the terminal groups R3 and R4 are independently an alkyl or alcoxy or alkylcarbonato group, each containing from 1 to 15 carbon atoms. The concentration of compounds 13-21 in the composition ought to be below 25%, preferably from 3% to 20%.

| | Acronym |
|---|---|
| 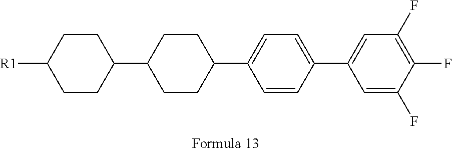 | 13n |
| 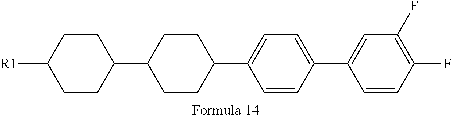 | 14n |
| 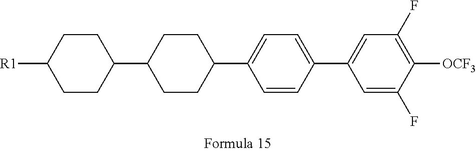 | 15n |
| 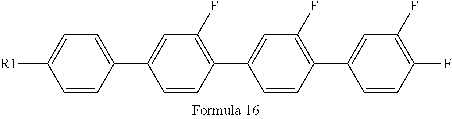 | 16n |
| 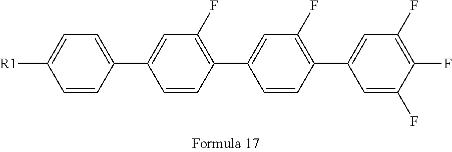 | 17n |
| 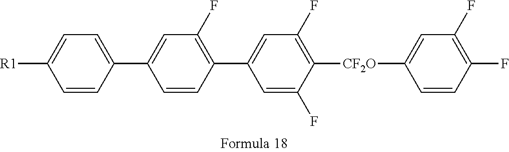 | 18n |
| 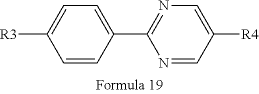 | 19n-m, 19n-Om, 19nO-m, 19nOCOO-m, 19n-OCOOm, 19nO-Om, 19nO-OCOOm, |
| | 19nOCOO-Om |
| 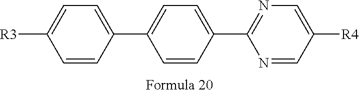 | 20n-m, 20n-Om, 20n-OCOOm, 20nO-Om, |
| 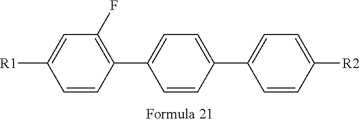 | 21n-m, 21n-Om 21n-OCOOm |

The compounds 13-18 have acronyms: 13n, 14n, 15n, 16n, 17n, 18n, respectively. The compound 19 has acronyms: 19n-m, 19n-Om, 19n-OCOOm, 19nO-m, 19nOCOO-m, 19nO-Om, 19nOCOO-Om, respectively for different terminal chains.

The compound 20 has acronyms: 20n-m, 20n-Om, 20n-OCOOm, 20nO-Om.

The useful compound 21 has acronyms: 21 n-m, 21 n-Om, 21 n-OCOOm.

Some pyrimidine derivatives expressed by the formulae 19 and 20 are new compounds prepared specially for the present disclosure.

The said new compounds are described in following way:

pyrimidines expressed by the formulae 19 and 20, wherein a terminal substituent R3 means alkylcarbonato group (H₂ₙ₊₁CₙOCOO) and right terminal substituent R4 means an alkyl group (CₘH₂ₘ₊₁) or alcoxy group (OCₘH₂ₘ₊₁) or left substituent R3 means an alkyl group (CₙH₂ₙ₊₁) or an alcoxy group (H₂ₙ₊₁CₙO) and right substituent R4 means alkylcarbonato group (OCOOCₘ H₂ₘ₊₁), in which symbols n and m denote independently numbers 1 to 15.

When a further decrease of the threshold voltage is needed, to the invented composition a limited amount of cyanocompounds may be added.

The composition modified in this way contains additionally at least one liquid crystalline cyanocompound selected from analogues of compounds 4-12, wherein the terminal fluorine atom is replaced by the cyano group (F→CN). The concentration of compounds with the terminal cyano group ought to be below 25 wt. %, preferably below 20 wt. % to keep the monolayer structure of the SmA phase. These compounds have acronyms: 4n-CN-12n-CN.

This aspect of the present disclosure is described in the following way: the smectic A₁ composition according the present disclosure modified in such way that it contains additionally at least one cyano compound, preferable a fluorinated cyano compound, selected from the set of analogues of compounds 4-18, wherein the terminal fluorine atom was replaced by the cyano group (CN), in the amount below 25 wt %, preferable in the amount below 20 wt %.

The invented fluorinated smectic A composition shows a small solubility of quaternary ammonium salts commonly used to increase conductivity of the smectic composition to a level needed to observe the dynamic scattering effect.

In order to increase of conductivity of the smectic A composition to a desired level of 10⁻⁹ Ohm⁻¹cm⁻¹ or higher it is proposed to use a new kind of ionic compounds, which have higher solubility and a higher degree of dissociation in comparison to quaternary ammonium salts. The ionic compounds not only increase the concentration of free ions in the liquid crystalline composition, but also fulfills a role of a surface active agent promoting the homeotropic orientation of molecules towards the electrodes.

This aspect of the present disclosure is described in following way:

the smectic A composition according to the present disclosure modified in such way that it contains additionally at least one or more ionic dopants with a delocalized charge of the cation selected from ionic compounds expressed by the general formulae 22-26.

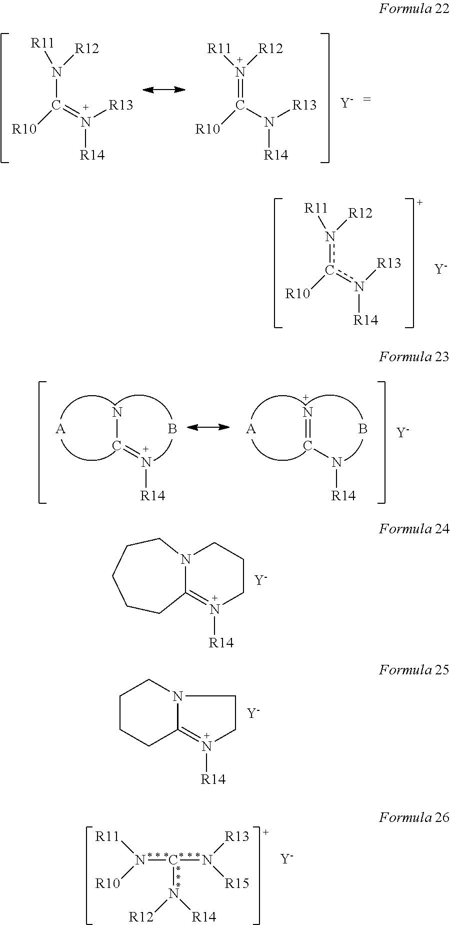

wherein substituents R10, R11, R13 and R15 in the cationic part of molecule denotes independently a hydrogen atom or an alkyl group or an allylcyclohexyl group or a phenyl group or an alkylphenyl group, and the alkyls may be the same or different and contain from 1 to 25 carbon atoms; the substituents R12 and R14 each denotes an alkyl group containing independently from 1 to 25 carbon atoms, profitable from 12 to 18 carbon atoms; in the formula 23 symbols A and B denote a part of the ring containing from two to five methylene groups wherein specially preferable compounds are those with in A four or five methylene groups and in B three or two methylene groups presented by general formulae 24 (A=5, B=3) and 25 (A=4, B=2), wherein R14 means preferably alkyl group containing from 2 to 18 carbon atoms; the anion Y⁻ denotes an organic or an inorganic anion preferably: Cl⁻ (a), Br⁻ (b), ClO₄⁻ (c), CF₃SO₃⁻ (d), R5-SO₃⁻ (e), SON⁻ (f),

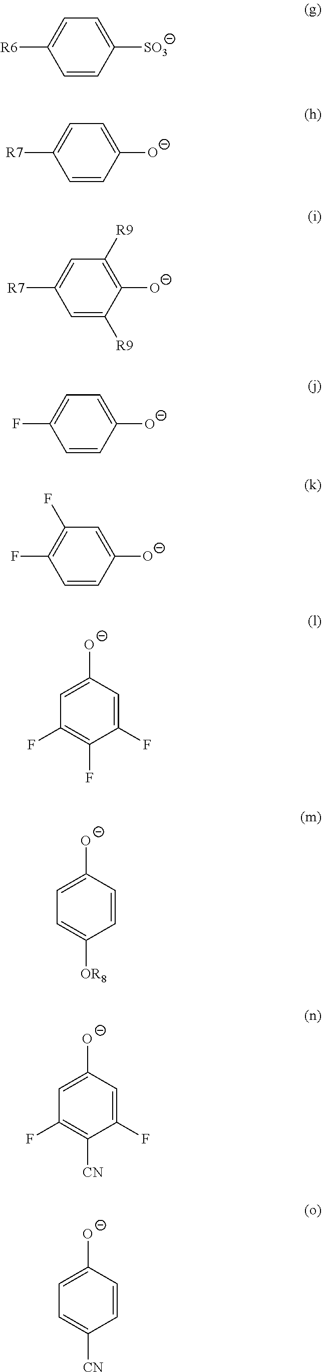

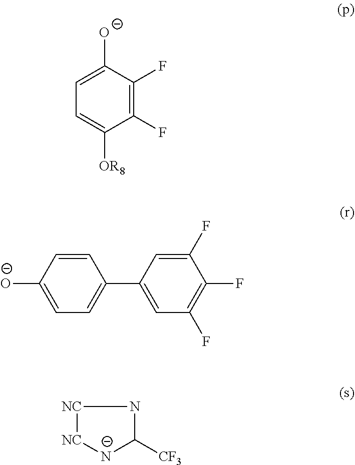

wherein substituents R5, R6, R7 and R8 mean a hydrogen atom or a normal branched alkyl group or an iso-alkyl group or a sec-alkyl group each containing independently from 1 to 20 carbon atoms, and R9 means a branched alkyl group, profitably a tert-butyl group.

The amidinium salts (expressed by the formulae 22-25) are quite different ionic compounds from ammonium salts. The first ones are derivative of carboxylic acids (R10COOH) while the second ones are derivative of tertiary amines (R11R12R13N).

The amidinium salts dissociate more easily in less polar medium and form more weakly bonded ionic parts than in ammonium salts, because positive charge of the cation is delocalized between three atoms [N—C—N]⁺, their structure is described by two resonance form, see formulae 22 and 23.

A specially high conductivity may be expected for ionic compounds wherein the charge of anion is delocalized, as in case of anions denoted with letters g-s.

The mentioned ionic compounds are better soluble in the liquid crystal composition and their anions show higher mobility, because they are weakly bonded in ionic pairs.

According to this idea a similar nature may show guanidinium salts (formula 26) which a hybrid structure may be presented by the three resonance structures responsible for the delocalization of the cation charge among four atoms.

Crown ethers, which are cyclic polyethers, also form delocalized cations in their complexes with inorganic or organic salts.

This aspect of the present disclosure is described in the following way: the smectic A composition according the present disclosure modified in such way, that it contains at least one or more ionic dopants with a delocalized charge of the cation selected from ionic compounds being crown ether complexes formed from salts of alkaline metal preferably from potassium salts and crown ethers expressed by the general formulae 27 and 28, wherein symbol A means a cyclohexane ring or a benzene ring and substituent R16 mean independently a hydrogen atom or an alkyl group, the same or different length containing from 1 to 16 carbon atoms, and anion Y⁻ means an organic or an inorganic anion preferably the same as described earlier in the case of amidinium salts.

The complex salts of crown ethers may be used as ionic dopants independently or preferably together with amidinium salts.

Some of amidinium salts were proposed earlier as ionic solvents for carrying out chemical reactions or using in chemical processes, for example for separation of cellulose from a wood (A. Diop, Bioresource, 8, 4270-82 (2013); D. Dandle et al. U.S. Pat. No. 8,049,120). The salts formed directly from an amidine and inorganic acids were used in that cases.

Proposed by us amidinium and guanidinium salts with the formulae 22 and 26 are especially convenient for the increase of orientation and conductivity of fluorinated liquid crystal compositions. They can play the same role in the case of other smectic A compositions, therefore they are included to the present disclosure in the following way:
an ionic compound expressed by the general formulae 22-26

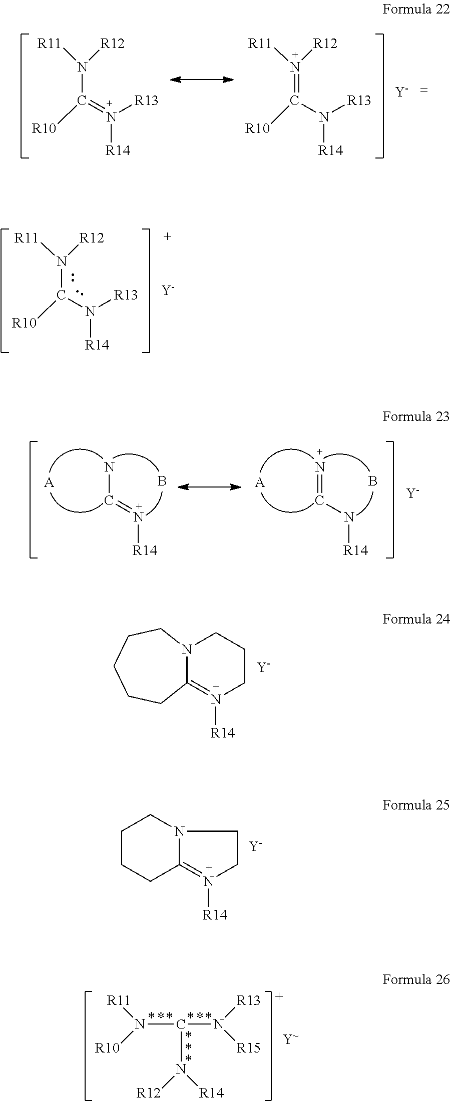

wherein substituents R10, R11, R13 and R15 in cationic part of the molecule are meaning independently a hydrogen atom or an alkyl group or an alkylcyclohexyl group or a phenyl group or an alkylphenyl group and they are the same or different and contain 1 to 25 carbon atoms; the substituents R12 and R14 are an alkyl group containing independently from 1 to 25 carbon atoms, preferably from 12 to 18 carbon atoms; in the formula 23 symbols A and B denote a part of the ring containing from two to five methylene groups leading to specially preferable compounds with the system of rings expressed by formula 24 (A=5, B=3) or 25 (A=4, B=2), wherein R14 is preferably an alkyl group containing from 2 to 18 carbon atoms; the anion Y⁻ denotes an organic or an inorganic anions preferably: Cl^{θ}— (a), Br^{θ}— (b), ClO₄^{θ}— (c), CF₃SO₃^{θ}— (d), R5-SO₃^{θ}— (e), SCN^{θ} (f)

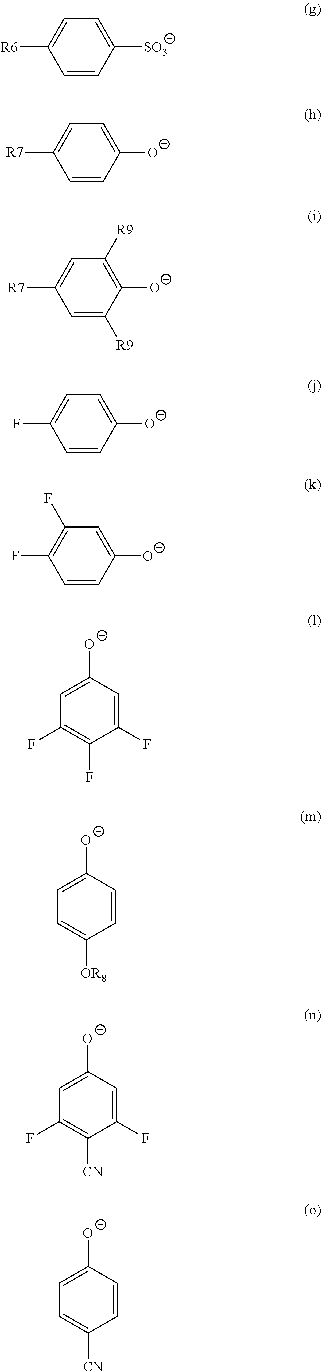

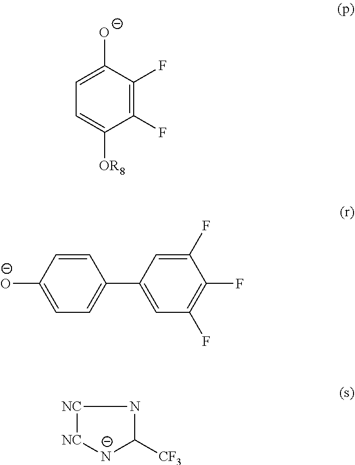

wherein substituents R5, R6, R7 and R8 are meaning a hydrogen or an normal alkyl group or an iso-alkyl group or a sec-alkyl group each containing independently from 1 to 20 carbon atoms and R9 is meaning a branched alkyl group preferably the tert-butyl group used as an ionic dopant to increase conductivity in smectic A compositions.

In the similar way we included ionic compounds expressed by general formulae 27 and 28 to the present disclosure:

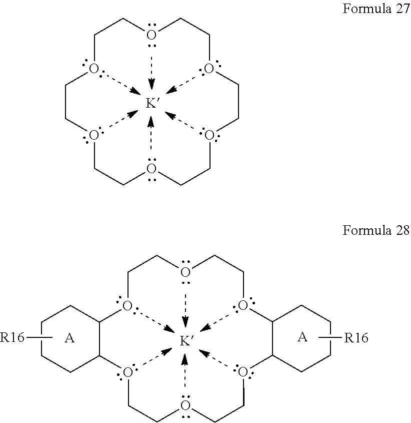

wherein symbol A is a cyclohexane ring or a benzene ring and substituents R16 are meaning independently a hydrogen atom or an alkyl group the same or different containing each from 1 to 16 carbon atoms; anion Y⁻ is an organic anion or an inorganic anion preferably: Cl^{θ—} (a), Br^{θ}— (b), ClO₄^{θ}— (c), CF₃SO₃^{θ}— (d), R5-SO₃^{θ}— (e), SCN^{θ} (f)

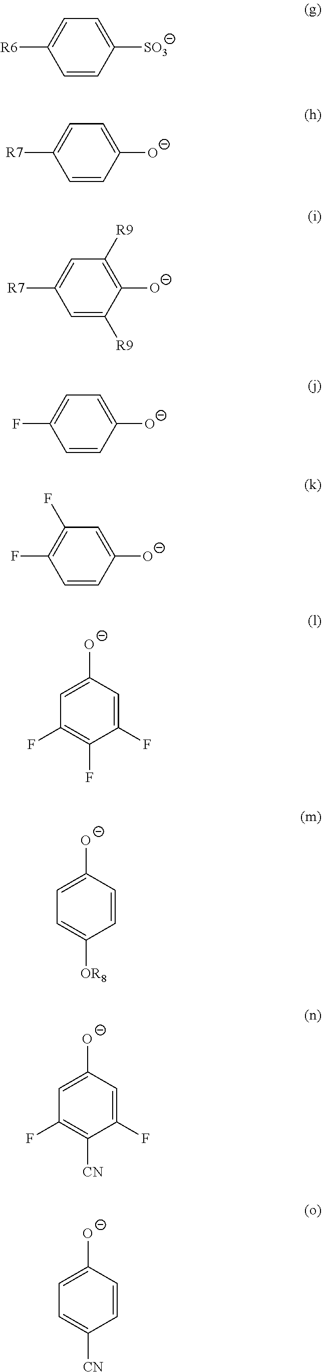

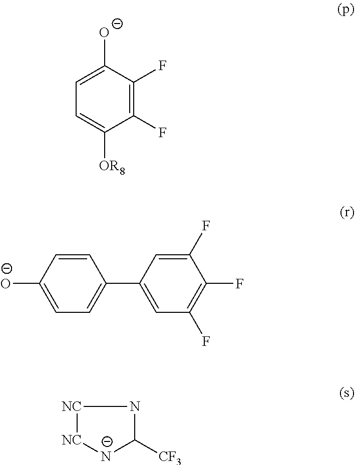

used as an ionic dopant to increase conductivity in smectic A compositions.

For obtaining a color picture or a color background to the invented composition may be added: an ionic dye or a nonionic dye with a property of an isotropic or an anisotropic absorption of light.

We propose to use a composition according the present disclosure which contains additionally at least one dye or a few dyes (when black picture is desired) with dichroic property (anisotropic absorption) or isotropic properties with an ionic or nonionic structure, preferably dyes expressed by the formulae 34 or 35 or an antrachinone dye preferably blue dye AB4 produced by Nematel company in the concentration below 3 wt. %.

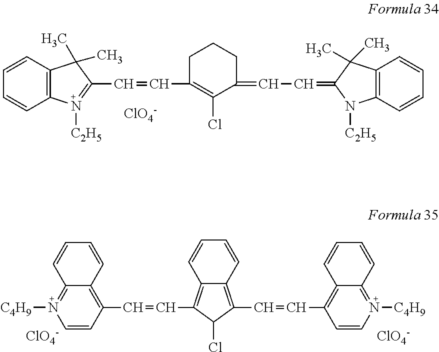

The method of obtaining of smectic A composition doped with ionic compounds characterized in that: said smectic A composition composed of compounds selected from the set of compounds 1-21 is dissolved in an organic solvent, preferably in fluorobenzene or chloroform, and the selected ionic compounds are dissolved in an organic solvent, preferably in fluorobenzene or chloroform, then both solutions are combined, carefully mixed and filtrated by a micropore membrane of pore diameter d<2 μm. Then the solvent is evaporated at the beginning under normal pressure and next at low pressure to remove solvent completely, successively the composition is heated in the stream of an inert gas, the concentration of the ionic compound is taken in the amount to obtain conductivity of order of 10⁻⁹ Ohm⁻¹cm⁻¹ or higher.

The described procedure leads to removing oxygen from the composition. The presence of dissolved oxygen influences unfavorably on a thermal and photochemical stability of liquid crystal composition and its lifetime. Therefore, after the evaporation of the solvent is preferably to heat the composition in an inert gas for several hours. When the liquid crystal mixture is used to fill display panels, as recommended inert gas may be pure nitrogen or pure argon. The same inert gas may be used also when the liquid crystal composition is dedicated for smart window panels, but here in some case using of the krypton may be more advantageous. It is preferable to store the liquid crystal composition in an inert gas atmosphere and to carry out the filling process in a chamber filled with an inert gas or in vacuum. The photochemical reactions of radical character are mainly responsible for shortening of the life time of smart windows.

From the state of the art many stabilizers are known, which have ability to inhibit radical reactions initiated by photolysis of liquid crystal compounds. Alkyl radicals formed by breaking C—C bonds are transformed into peroxides which accelerate further degradation process. Radical stabilizers were often added to polymers and also to the nematic composition, see Patent Appl. WO 2009/115226A1.

Therefore we propose to modify the method of obtaining of the smectic A composition mentioned above in following way: to the solution containing dissolved liquid crystal composition adds additionally a compound with ability to inhibit radical reactions, preferable from a phenol family expressed with the formula 29, wherein substituents R9 in orto-position to hydroxyl group is a bulky group, preferable tert-butyl group or amine derivatives expressed with the formulae 30-33, wherein substituent R17 denotes preferable a hydrogen atom or a benzyl group or a benzoic group.

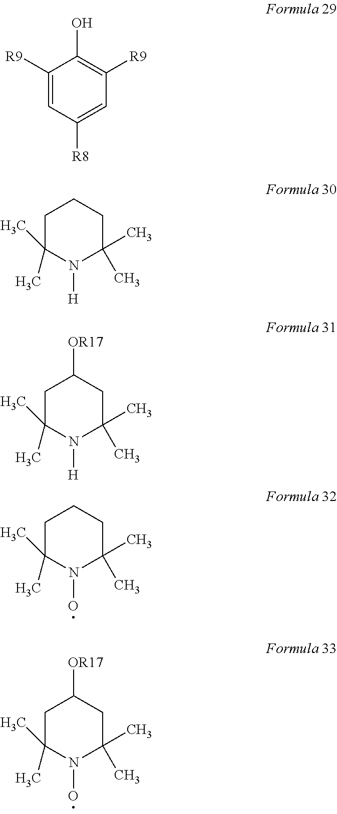

The composition obtained by the above mentioned methods is especially suitable to produce smart windows with a privacy function or displays with memory, which consists of two glass or plastic substrates with electrodes and orienting layers, wherein both electrodes are transparent or one contains an evaporated reflective metal layer, preferable from aluminum, and the gap between them of 5 to 20 μm is filled with the smectic A composition doped with ionic compounds which the composition and the method of preparation were described above.

The below mentioned examples illustrate in details all aspects of the disclosed embodiments, but do not limit its range. The all concentrations in examples are given in wt. % and values of thermodynamic parameters of compounds such as the melting point and the melting enthalpy are given in ° C. and kJ/mol, respectively.

The glass cells with a gap of 15 μm filled with the liquid crystal composition were used to measure conductivity and such a parameters of the DS effect as threshold voltage and saturation voltage. Transparent electrodes were formed from indium-tin oxide (ITO) of specific resistivity 100 Ohm/cm² and with surface 1.27×1.27 cm². The orienting layer providing a homeotropic orientation of the molecular director of the smectic composition was obtained using a thin layer of SE1211 (by Nissan Chem.) polyimide, which was deposited on ITO electrodes with a spincoating method, then dried and cured. The testing cells were filled with the liquid crystals composition from the isotropic phase, and they were cooled to the room temperature (RT) and closed.

### Example 1

Suitable Compounds for Obtaining of the Composition

In Table 1, single compounds and their phase transition temperatures (° C.) and melting enthalpies (kJ/mol) are listed, that were selected as components especially preferable for obtaining of the smectic A composition according to the present disclosure.

Symbols n and m mean number of carbon atoms in alkyl chains CₙH₂ₙ₊₁ and CₘH₂ₘ₊₁ at left and right side of the molecule, respectively.

TABLE 1
List of preferable compounds for the obtaining of the composition
with smectic A₁ phase and their temperatures (° C.) of the phase transitions
and the melting enthalpies ΔH°ₘ (kJ/Mol).

| No. | Acronym | n | m | Phase transition temperatures [° C.] | ΔH°ₘ |
|---|---|---|---|---|---|
| 1. | 1n-Om | 5 | 6 | Cr 38.2 Iso | 18.5 |
| 2. | 1n-Om | 5 | 7 | Cr 42.2 Iso | 36.9 |
| 3. | 1n-Om | 5 | 8 | Cr 34.9 SmA 38.2 N 39.0 Iso | 34.9 |
| 4. | 2n-OCOOm | 5 | 3 | Cr 88.3 SmA 123 N 194.8 Iso | |
| 5. | 2n-OCOOm | 5 | 5 | Cr 88.3 SmA 123.0 N 194.8 Iso | |
| 6. | 2n-m | 3 | 2 | Cr 76.6 N 135.6 Iso | |
| 7. | 2n-Om | 1 | 3 | Cr 86.7 N 188.5 Iso | 26.4 |
| 8. | 2n-m | 3 | 4 | Cr 56.5 (SmA 52) N 133.5 Iso | |
| 9. | 2n-m | 3 | 5 | Cr 64.5 SmA 113 N 128.5 Iso | |
| 10. | 2n-m | 5 | 7 | Cr 41.2 SmA 116.1 N 131.2 Iso | |
| 11. | 3n-m | 5 | 5 | Cr 35 SmA 115 N 118 Iso | |
| 12. | 3n-OCOOm | 3 | 2 | Cr 89.0 SmA 118.5 N 166.6 Iso | |
| 13. | 3n-OCOOm | 3 | 3 | Cr₁ 103.0 Cr 77.5 SmA 109.5 N 153.1 Iso | 15.4; 8.4 |
| 14. | 4n | 5 | | liquid | |
| 15. | 4n-CN | 5 | | liquid | |
| 16. | 5n | 3 | | Cr 85.0 SmA 94.5 N 132.5 Iso | 21.1 |
| 17. | 5n | 4 | | Cr 70.1 SmA 96.8 N 116.5 Iso | 18.3 |
| 18. | 5n | 5 | | Cr 75.8 SmA 104.75 N 126.2 Iso | 18.5 |
| 19. | 5n-CN | 5 | | Cr 82 N 143 Iso | |
| 20. | 5n | 7 | | Cr 69.6 SmA 105.2 N 118.6 Iso | 24.0 |
| 21. | 5n | 9 | | Cr 64.4 SmA 71.2 N 80.9 Iso | |
| 22. | 6n | 5 | | Cr₁ 48.8 Cr 94.0 SmA 97 Iso | 8.0; 7.9 |
| 23. | 6n-CN | 5 | | Cr 103 N 120 Iso | |
| 24. | 7n | 3 | | Cr 60.7 Iso | 17.4 |
| 25. | 8n | 2 | | Cr 99.6 Iso | 25.7 |
| 26. | 8n | 3 | | Cr 72.7 N 85.1 Iso | 25.0 |
| 27. | 8n | 4 | | Cr 58.7 SmA 74.3 N 85 Iso | 19.2 |
| 28. | 8n | 5 | | Cr 64.2 SmA 72.4 N 86.4 Iso | 29.4 |
| 29. | 8n-CN | 5 | | Cr 82.5 N 162.3 Iso | |
| 30. | 9n | 3 | | Cr 62.3 Iso | 21.0 |
| 31. | 10n | 5 | | Cr 85.3 SmA 99.3 N 160.1 Iso | 18.14 |
| 32. | 11n | 5 | | Cr 72.8 N 146.4 Iso | 19.6 |
| 33. | 12n | 3 | | Cr 67.9 N 98.6 Iso | 24.4 |
| 34. | 12n | 5 | | Cr 55.1 N 108.2 Iso | 18.1 |
| 35. | 13n | 3 | | Cr 103.1 N 260.0 Iso | 17.6 |
| 36. | 13n | 5 | | Cr₁ 73.6 Cr2 88.4 N 250.5 Iso | 19.0; 16.8 |
| 37. | 14n | 2 | | Cr 91.0 SmA 115.43 N 275 Iso | 18.3 |
| 38. | 14n | 5 | | Cr 75.0 SmA 97.0 N 285 Iso | 31.6 |
| 39. | 15n | 2 | | Cr 93.9 N 250.0 Iso | 21.6 |
| 40. | 15n | 5 | | Cr 80.6 N 268.0 Iso | 13.9 |
| 41. | 16n | 5 | | Cr 114.5 SmA 195.0 N 235.6 Iso | 35.7 |
| 42. | 17n | 5 | | Cr 101.8 SmA 167.5 N 201.6 Izi | 33.7 |
| 43. | 18n | 5 | | Cr 44.5 SmA 90.5 N 140.7 Iso | 31.2 |
| 44. | 19nO-Om | 4 | 8 | Cr 58 SmC 85 SmA 95 N 98 Iso | 16.3 |
| 45. | 19nO-Om | 6 | 8 | Cr 44.5 SmC 89.8 SmA 97.4 N 99.5 Iso | 29.1 |
| 46. | 19nO-m | 6 | 8 | Cr 27.5 SmC 46.5 SmA 57.4 N 65.5 Iso | 24.2 |
| 47. | 19nOCOO-m | 7 | 9 | Cr 35.7 SmA 38.8 N 49.5 Iso | 18.9 |
| 48. | 19nOCOO-m | 5 | 9 | Cr 42.6 SmC 58.0 SmA 78.9 N 79.5 Iso | 32.0 |
| 49. | 19nOCOO-m | 9 | 9 | Cr₁ 52.3 Cr2 58.3 SmC 69.1 SmA 77.9 N 81.4 Iso | 24.4; 22.9 |
| 50. | 19nOCOO-m | 10 | 9 | Cr₁ 43.1 Cr2 57.4 SmC 70.6 SmA 78.1 N 81.6 Iso | 19.6; 22.3 |
| 51. | 20n-m | 7 | 5 | Cr₁ 59.3 Cr2 55.8 SmC 132.8 N 147.8 Iso | 31.4 |
| 52. | 20n-m | 5 | 6 | Cr₁ 44.5 Cr2 51.1 SmC 72.8 N 157.9 Iso | 29.0 |
| 53. | 20n-Om | 5 | 8 | Cr 98 SmC 159 N 186.9 Iso | |
| 54. | 20n-OCOOm | 7 | 8 | Cr 93.3 SmA 131.7 N 169.8 Iso | |
| 55. | 21n-m | 5 | 5 | Cr 72.5 SmC 80.0 N 136.0 Iso | |
| 56. | 21n-Om | 5 | 8 | Cr 89.0 SmC 119.0 N 158.0 Iso | |
| 57. | 21n-OCOOm | 5 | 3 | Cr 67.6 SmC (66.7) N 168.3 Iso | 26.5 |

value in parenthesis ( ) means a monotropic phase

### Example 2

### Synthesis of the Compounds 1-21

The majority of compounds listed in Table 1 presented in Example 1 are the homologues selected from known families, but only some of them were prepared earlier and methods of their preparation were only partially described. The compounds 1n-m, 1n-Om, 2n-m, 2n-Om, 3n-m, 3n-Om, 5n, 7n, 8n, 9n, 21n-m and 21n-Om were prepared by inventors in the way shown in the Scheme 1. The compounds 4n, 6n, 17n were prepared by inventors in the way shown in the Scheme 2.

The Suzuki-Miyaura coupling reaction of a halogenoaryl (use of a iodoaryl derivative is preferred) with a phenyl boronic acid in the weak alkaline medium and water-acetone mixture at the presence of a palladium catalyst was used. Additionally, diadamantyl-n-butyl hydrophosphonium bromide as co-catalyst was necessary to use to increase the reaction speed in the case of the such reactants as bromoaryls.

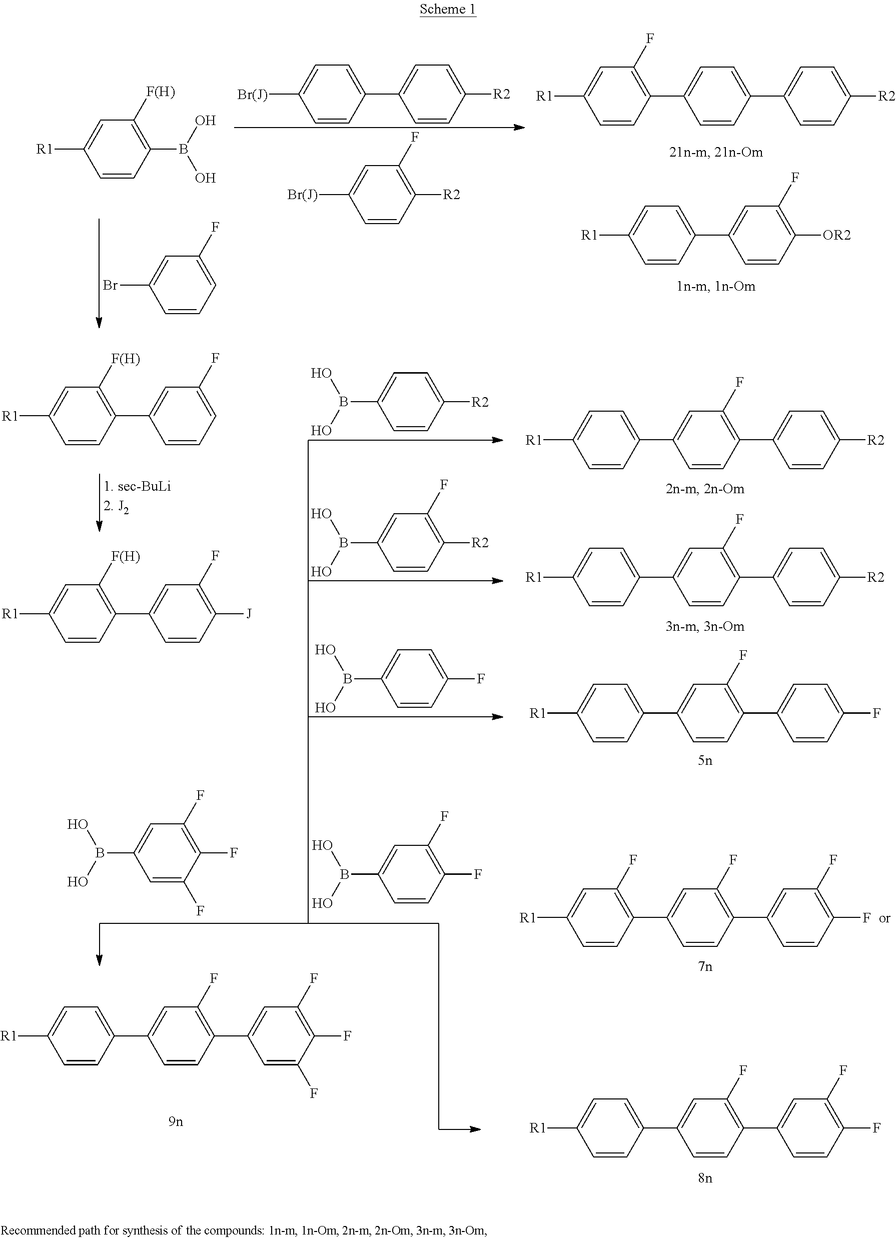

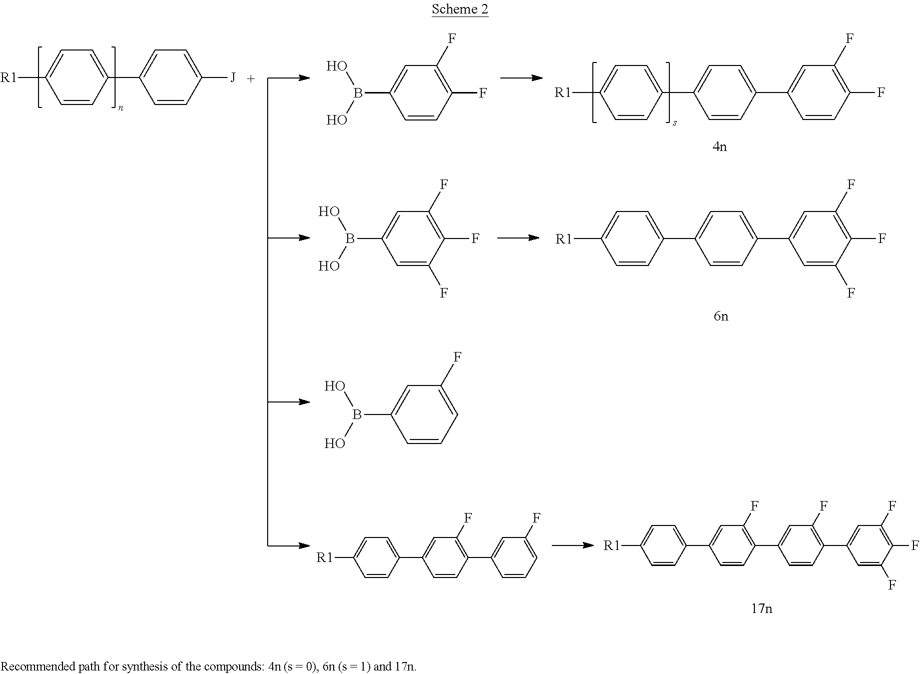

The compound 16n was prepared analogously as the compound 17n. In the both cases the stage of coupling of alkyl iodobenzene (s=0) with 3-fluoroboronic acid was repeated twice (the second coupling was preceded by the reaction of iodination) and the intermediate product 4″-alkyl-2′,3-difluoroterphenyl was formed, which in the following reaction of iodination and then the reaction of coupling with 3,4-difluorophenyl boronic acid or 3,4,5-trifluorophenyl boronic acid was transformed into the compound 16n or 17n.

The compounds 13, 14 and 15 were prepared in analogous way as it was described in the work published earlier: R. Dqbrowski et al., Liq. Cryst., 40, 1339-13 (2013). Compound 18 was a commercial product (by Vailant-China).

The compounds 19 and 20 and also 11 and 12 were prepared by the pathway shown on Scheme 3.

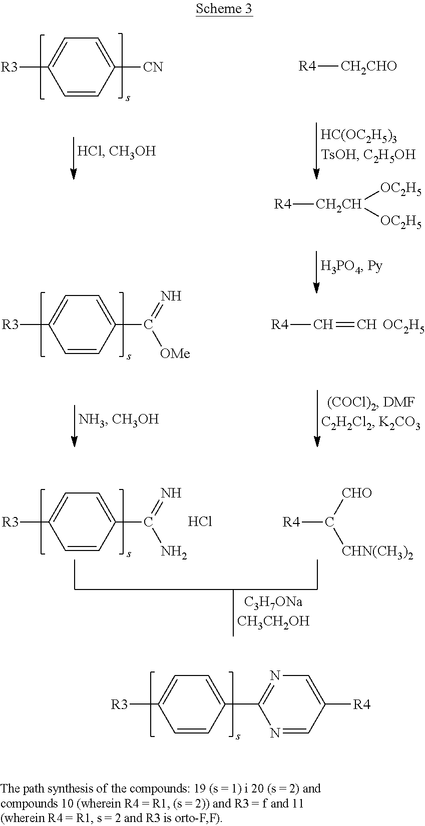

The signs on Scheme 3 are as follow: s=1 denotes compound 19, s=2 denotes compound 20. Compounds 19 and 20 in which R3 or R4 denote carbonato group H₂ₙ₊₁CₙOCOO or OCOOCₘH₂ₘ₊₁ are new compounds, and they are included in the scope of the present disclosure. These compounds were synthesized from compounds 19nO-m and 19n-Om or 20nO-m and 20n-Om using them as intermediate compounds in which the ether bond CₙH₂nO-phenyl or CₘH₂mO-phenyl was cleaved and the formed phenol was then treated by a suitable alkyl chloroformate.

Synthetic procedure shown on Scheme 1 is given in detail on the example of the compound 5.3 belonging to the homologous series 5n.

### Stage 1. 4′-Propyl-3-fluorobiphenyl

3-fluorobromobenzene (122 g, 0.697 mol), 4-propylphenylboronic acid (114.3 g, 0.697 mol), anhydrous K₂CO₃ (385 g, 2.79 mol), acetone (500 cm³) and water (500 cm³) were placed in a flask (2.5 l), stirred and heated slowly under nitrogen flow to obtain boiling temperature (60° C.). The reaction mixture was refluxed yet for 0.5 h. Then the temperature was decreased to 50° C. and catalysts: di-(1-adamantyl)-n-butylhydrophosphonium bromide (10 mg) and palladium acetate (10 mg) were added. An exothermic reaction was starting and temperature of the boiling point was reached soon. The mixture was kept at the refluxing state for an hour and then the acetone was evaporated. The rest was diluted with water and next the hexane (500 cm³) was added. The obtained two-phase system was stirred energetically, then it was filtrated through a filter plate. The hexane layer was separated and dried over anhydrous MgSO₄, which was then removed by filtration and hexane was evaporated from the solution on an rotary evaporator. The raw product was distilled under low pressure (0.5 mmHg).

The fraction, boiling at the range of 113-114° C., was collected.

Yield: 130.8 g, 0.611 mol (87.7%), the liquid product of the purity >99.9%.

### Stage 2. 4′-propyl-3-fluoro-4-iodobiphenyl

The 4′-propyl-3-fluorobiphenyl (130.8 g, 0.611 mol) prepared above and anhydrous THF (500 cm³) were placed in a flask and that solution was stirred and cooled to (−70° C.) during slow nitrogen flow. At this temperature the solution of sec-butyl lithium (436.4 cm³, 0.611 mol) in cyclohexane with concentration 1.4 mol/dm³ was added by dropping. After dropping of the total amount of the sec-butyl lithium solution the reaction mixture was stirred further at (−70° C.) for two hours and while keeping this temperature a solution of iodine (155.2 g, 0.611 mol) in THF (500 cm³) was added by dropping. Stirring at temperature (−70° C.) was continued for 0.5 hour and then cooling was stopped. When the mixture reached the room temperature a saturated water solution of sodium sulphate (IV) was added until an excess of iodine was removed (to discoloration).

Then THF was evaporated on a rotatory evaporator. The residue was diluted with water and extracted with toluene (500 cm³). The toluene solution was twice washed with water, than with sodium sulphate (IV) solution, and subsequently with water (three times) and finally dried over anhydrous MgSO₄. Magnesium sulphate was filtrated off, and toluene was distilled of on a rotary evaporator. The residue was crystallized from ethanol (1 dm³).

Yield: 180 g, 0.532 mol (87.1%).

### Stage 3. 4″-Propyl-2′,4-difluoroterphenyl (compound 5.3)

The 4′-propyl-3-fluoro-4-iodobiphenyl (34 g, 0.1 mol), 4-fluorophenylboronic acid (14 g, 0.1 mol), K₂CO₃ (52.2 g, 0.4 mol), acetone (100 cm³) and water (100 cm³) were placed in the flask (0.5 dm³) and the mixture was stirred and heated under nitrogen to reach the boiling temperature (60° C.) and it was refluxing yet for 0.5 hour. Then the temperature was decreased to 50° C. and palladium acetate (10 mg) was added. The exothermic reaction was starting and temperature was reaching the boiling point. The reaction mixture was kept yet at the refluxing state for an hour and then acetone was distilled off. The residue was diluted with water and hexane was added (250 cm³). The obtained two phase system was stirred energetically. The hexane layer was separated and it was washed with water (three times) and it was dried over anhydrous MgSO₄, which was next removed by filtration. The hexane solution was filtrated through a silica gel layer and hexane was evaporated. The solid residue was crystallized from ethanol (240 cm³). Yield: 29.9 g, 0.089 mol (89%), the phase transitions: Cr, 85; Sm A, 94.5; N, 132.5; Iso.

The 4″-propyl-2′,3,4-trifluoroterphenyl (compound 8.3; the phase transitions: Cr, 72.7; N, 85.1; Iso) was obtained analogously, when the 3,4-difluorophenylboronic acid was used instead of the 4-fluoroboronic acid.

The 4″-propyl-2′,3,4,5-tetrafluoroterphenyl (compound 9.3; the melting point 62.3° C.) was obtained analogously, when the 3,4,5-trifluorophenyl boronic acid was used instead of the 4-fluorophenyl boronic acid.

The 4′-propyl-3-fluorobiphenyl was also a substrate to the preparation of compounds 16.3 and 17.3.

### Example 3

### Synthesis of Amidinium Salts.

The method of the preparation of amidinium salts is illustrated on the example of salts formulated from 1,8-diazabicyclo[5.4.0]undec-7-ene (DBU).

In the first stage 1-hexadecyl-1,8-diazabicyclo[5.4.0] undec-7-enium bromide was obtained by treating 1,8-diazabicyclo [5.4.0] undec-7-ene (DBU) with 1-bromohexadecane. The prepared amidinium bromide was used as a ionic dopant (its acronym is II.16b) or it was used as an intermediate compound for the preparation of quaternary amidinium salts with other anions. In this case amidinium bromide was transformed successively to amidinium hydroxide (second stage) which in the reaction with an organic or inorganic acid (third stage) yield an amidinium salt with a desired anion. The prepared ionic compounds are denoted with the acronym 1.nYm, wherein n denotes the number of carbon atoms in R14 chain, Y⁻ means an anion and m denotes number of carbon atoms in anion chain R5 or R6 or R7 or R8. Their formulae are listed in Table 2.

### Stage 1. 1-hexadecyl-1,8-diazabicyclo[5.4.0]undec-7-enium bromide [C₁₆H₃₃DBU]⁺Br⁻, compound II.16b

A solution prepared from cyclohexane (50 cm³) and DBU (15 cm³, 0.1 mol) was heated to 45° C. and at this temperature 1-bromohexadecane (30 cm³, 0.1 mol) was added by dropping during 1 hour. Heating was continued for 5 hours. Then the reaction mixture was left to cool to the RT. After 12 hours the obtained crystalline solid was separated by filtration and then it was recrystallized from ethyl acetate.

Yield: 27.1 g (59%). Colorless crystals, m.p. 45-48° C. The compound is well soluble in methanol and chloroform.

In analogous way 1-ethyl-1,8-diazabicyclo[5.4.0] undec-7-enium bromide (C₂H₅-DBU)+Br, compound II.2b was obtained.

Yield: 26 g (98%), colorless crystals. The compound is well soluble in ethanol and ethyl acetate.

### Stage 2. 1-Hexadecyl-1,8-diazabicyclo[5.4.0]undec-7-enium hydroxide [C₁₆H₃₃-DBU]⁺OH⁻

[C₁₆H₃₃-DBU]⁺Br (4.6 g, 0.01 mol) was solved in propanol (120 cm³) and to this solution Ag₂O (1.73 g, 0.0075 mol) was added. The reaction mixture was intensively stirred and heated for 8 hours at temperature 60° C. Then it was cooled to RT and AgBr and excess of Ag₂O was filtered off and the liquid was evaporated to dryness on rotary evaporator. The solid crystalline product was obtained, yield 3.8 g (91%). The compound is well soluble in ethanol and chloroform.

### Stage 3. 1-Hexadecyl-1,8-diazabicyclo[5.4.0]undec-7-enium chlorate (VII), [C₁₆H₃₃-DBU]⁺ClO₄⁻ (compound II.16c)

The [C₁₆H₃₃-DBU]⁺OH⁻ (2.3 g, 0.0058 mol) was dissolved in methanol (30 cm³) and then the chloric (VII) acid was added in the amount to obtain pH=7 (about 0.5 cm³ of the acid with the concentration of 65 wt % was needed). Then the solvent was evaporated on rotary evaporator. Yield 2.5 g (90%), the solid with yellowish color. It was recrystallized from hexane. The compound is well soluble in methanol and ethyl acetate.

In analogous way were obtained:
1-hexadecyl-1,8-diazabicyclo[5.4.0]undec-7-enium chloride (II.16a),
1-ethyl-1,8-diazabicyclo[5.4.0]undec-7-enium 1,1,1-trifluoromethanesulphonate (II.2d),
1-hexadecyl-1,8-diazabicyclo[5.4.0]undec-7-enium 1,1,1-trifluoromethanesulphonate (II.16d), white crystal product,
1-ethyl-1,8-diazabicyclo[5.4.0]undec-7-enium dodecylsulphonate (II.2e12),
1-hexadecyl-1,8-diazabicyclo[5.4.0]undec-7-enium dodecylsulphonate (II.16e12),
1-hexadecyl-1,8-diazabicyclo[5.4.0]undec-7-enium 3,4-difluorophenolate (II.16k). The compound is light-yellow-orange liquid, yield 88%
1-hexadecyl-1,8-diazabicyclo[5.4.0]undec-7-enium 4-dodecylbenzenesulphonate (II.16g12). The compound is light-yellow liquid, yield 92%.

### Example 4

### Syntheses of complexes of 18-crown-6 ether with alkaline metal salts

The complex of 18-crown-6 ether with potassium 4-dodecylbenzenesulphonate (compound III.g12, Table 2).

The equimolar amounts of 18-crown-6 ether (0.53 g, 0.02 mol) and potassium dodecylbenzenesulphonate (0.73 g, 0.002 mol) in methanol (30 ml) were refluxed for 4 hours. Then methanol was evaporated to dryness. The viscous, colorless, clear liquid was obtained, yield 1.2 g.

In analogous way were obtained:

the complex of 18-crown-6 ether with potassium 3,4-difluorophenolate (compound III.k), viscous orange liquid,
the complex of 18-crown-6 ether with potassium bromide (compound III.b), white crystalline product,
the complex of 18-crown-6 ether with sodium dodecyl-1-sulphonate (compound V.e12), a viscous colorless liquid.

### Example 5

### Syntheses of the complexes of dibenzo-18-crown-6 ether with potassium salts

The complex of dibenzo-18-crown-6 ether with potassium chlorate (VII) (compound IVc).

The equimolar amounts of dibenzo-18-crown-6 ether (1.8 g, 0.005 mol) and potassium chlorate (VII) (0.69 g, 0.005 mol) in absolute ethanol (140 cm³) were refluxed for 7 hours when clear solution was obtained. The solution was cooled to 0° C. and the separated crystalline product was filtered off. Yield 1.7 g (68%), the crystalline compound, melting temp. 180° C.

Complex of dibenzo-18-crown-6 ether with potassium 4-dodecylbenzenesulphonate (compound IVg12).

Equimolar amounts of dibenzo-18-crown-6 ether (1.8 g, 0.005 mol) and potassium 4-dodecylbenzenesulphonate (1.8 g, 0.005 mol) in methanol (50 cm³) were refluxed for 4 hours. The hot solution was filtered and then cooled to temperature 0° C. Methanol was evaporated on a rotating evaporator. The residue was crystallized from ethyl acetate. The white crystalline product was obtained in amount of 2.8 g (77%), the melting point 85-88° C.

In analogous way were obtained:

the complex of dibenzo-18-crown-6 ether with potassium 3,4-difluorophenolate (compound IVk), yield 88%, the viscous liquid,
the complex of dibenzo-18-crown-6 ether with potassium 3,5-difluoro-4-cyanophenolate (compound IVn), yield 43%, the colorless liquid,
the complex of dibenzo-18-crown-6 ether with potassium 4-octylphenolate (compound IVh-8), yield 40%, the white crystalline product, crystallized from ethyl acetate,
the complex of dibenzo-18-crown-6 ether with potassium bromide (compound IVb), the white crystalline solid,
the complex of dibenzo-18-crown-6 ether with potassium dodecanyl-1-sulphonate (compound IVe12),
the complex of dibenzo-18-crown-6 ether with 1,1,1-trifluoromethyl-2-sulphonate (compound IVd).

### Example 6 (Comparative)

In this example the solubility and resistivity of solutions prepared from the ionic compound known from the state of the art as the preferable ionic dopants to smectic mixtures (hexadecyltrimethylammonium bromide—compound 1.16b) are compared with the solubility and resistivity of ionic compounds proposed in this present disclosure (see Table 2).

Fluorobenzene was used as solvent because it is chemically similar to the liquid crystalline composition used here.

The examination was made in the following way: always the same mole amount of a ionic compound (0.00004 mol) was placed in 2 cm³ of fluorobenzene. The mixture was mixed to dissolve the compound or to obtain equilibrium between the saturated solution and the solid. When a part of the compound was dissolved only, the sample of the saturated solution was taken (0.5 ml). The solvent was evaporated to dryness, the rest was weighted. Knowing the residue amount, the solubility was calculated.

The resistivity of the solution was measured by the bridge method using a Wyne Kerr Precision Component Analyzer 6425 and the measuring cells with electrodes of active area of 12.7 mm×12.7 mm. The electrodes were made of ITO. No orienting layers were deposited. The cell formed with two substrates with electrodes mentioned above was filled with the solution by capillary forces. The gap between electrodes was approx. 14.6 μm. The measured values of solubility and resistivity taken at frequencies of 50 Hz and 10 kHz are listed in Table 2.

TABLE 2
The comparison of solubility and resitivity (Ohm · cm) of ammonium and amidinium salts and complexes
salts of crown ethers solutions in C₆H₅F at temperature 25° C.

| | investigated ionic compound | | solubility in C₆H₅F | resistivity | |
|---|---|---|---|---|---|
| Acronym | cation | anion | mol/dcm³ | 50 Hz | 10 kHz |

ammonium salts

| | | | | | |
|---|---|---|---|---|---|
| I.16b | (CH₃)₃N⁺C₁₆H₃₃ | Br^{⊖} | 0.00274 | 5.0 · 10⁷ | 5.2 · 10⁷ |

amidinium salts

| | | | | | |
|---|---|---|---|---|---|
| II.2b | (C₂H₅DBU)⁺ | Br^{⊖} | 0.0077 | | |
| II.16b II.16c II.16d II.16k | 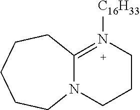 | Br^{⊖} ClO₄^{⊖} CF₃SO₃^{⊖} 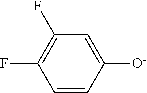 | >0.02 >0.02 >0.02 >0.02 | 7.8 · 10⁵ 1.0 · 10⁶ 8.7 · 10⁵ | 6.7 · 10⁵ 0.84 · 10⁶ 7.8 · 10⁵ |
| II.16n | | 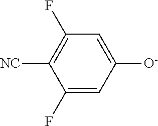 [SMILES: N#Cc1c(F)cc([O-])cc1F] | >0.02 | 3.9 · 10⁵ | 3.8 · 10⁵ |
| II.16g12 | | 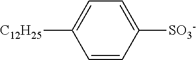 | >0.02 | 2.1 · 10⁶ | 1.9 · 10⁶ |

potassium complexes of crown ether

| | | | | | |
|---|---|---|---|---|---|
| IIIb IIIk | 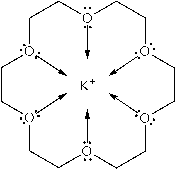 | Br^{⊖} 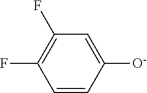 | 0.0104 >0.02 | | |
| III.g12 | | 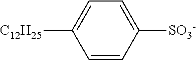 | >0.02 | | |
| IVb IVc IVk | 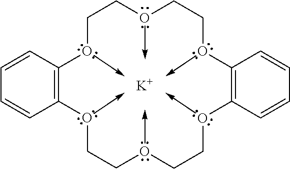 | Br^{⊖} ClO₄^{⊖} 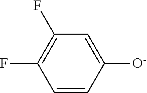 | 0.0042 <0.002 >0.02 | 4.2 · 10⁷ 1.1 · 10⁶ | 2.9 · 10⁷ 0.95 · 10⁶ |
| IVn | | 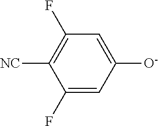 [SMILES: N#Cc1c(F)cc([O-])cc1F] | >0.02 | 4.2 · 10⁵ | 4.6 · 10⁵ |
| IVh8 | | 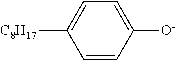 | 0.0107 | | |
| IVg12 | | 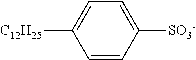 | >0.02 | 6.0 · 10⁵ | 4.5 · 10⁵ |

sodium complex of crown ethers

| | | | |
|---|---|---|---|
| V.e12 | 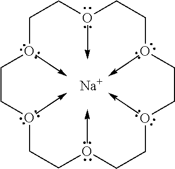 | C₁₂H₂₅SO₃^{⊖} | 0.019 |

The majority of investigated ionic compounds according to the present disclosure have solubility in fluorobenzene higher than 0.02 mol/dcm³ except of 1-ethyl-1,8-diazabicyclo[5,4,0]undec-7-enium bromide (compound II.2b) and complexes of 18-crown-6 ethers with KBr and KClO₄ (compounds IIIb and IVb and IVc, respectively). The commonly used hexadecyltrimethylamonium bromide (compounds I.16b) has solubility lower at least ten times. The proposed ionic dopants have resistivity lower than 2·10⁶ Ohm·cm except of complex dibenzo-18-crown-6 ether with KClO₄ (compound IVc), wherein resistivity is 4.2·10⁷ Ohm·cm, but also its resistivity is lower than observed for I.16b. Compound I.16b (hexadecyltrimethylammonium bromide) has resistivity 80 times higher than II.16b

(1-hexadecyl-1,8-diazabicyclo[5,4,0]undec-7-enium bromide) and 140 times higher than I.16n (1-hexadecyl-1,8-diazabicyclo[5,4,0]undec-7-enium 3,5-difluoro-4-cyanophenolate. The data in Table 2 show clearly, that the ionic compounds according to this present disclosure have solubility and conductivity in fluorobenzene much higher in comparison to the quaternary ammonium salts known from the state of the art.

### Example 7

### Undoped Composition.

Formulation of the smectic composition composed with two and three ring fluorinated compounds only.

The eutectic compositions were prepared from the compounds listed in Table 1. Their composition was calculated with using of a custom made computer program based on the equations:

$${lnx}_{k} = \frac{Δ {H}_{m}^{k}}{R} ( \frac{1}{T} - \frac{1}{{T}_{m}^{k}} )$$

$${\sum }_{k = 1}^{n} Xk = 1$$

wherein ΔH^{k}ₘ and T^{k}ₘ are melting enthalpy [J/mole] and melting temperature [K] of k—component, respectively, Xₖ—molar fraction, n—number of components, R—universal constant [8.315 JK⁻¹ mole⁻¹].

The calculated concentration in mole ratio was afterwards recalculated to wt. % with using of the same computer program. Since the multicomponent compositions crystallize difficulty therefore their melting points are not given. The calculated theoretical melting temperatures (eutectic points) are given for the compositions mentioned below.

Composition 1 (Four Components)

The composition comprising:

| Acronym of compound | wt % |
|---|---|
| 1.5-O6 | 34.12 |
| 1.5-O8 | 46.32 |
| 5.7 | 11.49 |
| 8.5 | 8.07 |

characterizes with the following phase transition sequence and temperatures: Cr<0° C.; SmA₁, 85.0-89.5° C.; Iso. The theoretical eutectic point is at (−0.87) ° C. Composition 1 in the whole range of the mesophase has smectic A₁ phase only, nevertheless that its three components: compounds 1.5-O8, 5.7 and 8.5, exhibit a nematic phase and two last ones even in a broad temperature range, see Table 1.

Composition 2 (Five Components)

The composition comprising

| Acronym of compound | wt % |
|---|---|
| 1.5-O6 | 31.40 |
| 1.5-O8 | 43.15 |
| 5.7 | 10.34 |
| 8.5 | 7.09 |
| 8.7 | 8.02 |

characterizes with the following phase transition sequence and temperatures: Cr<0° C.; SmA₁, 87-89° C.; Iso. The theoretical eutectic point is at (−3.5° C.).

Composition 3 (Five Components)

The composition comprising

| Acronym of compound | wt % |
|---|---|
| 1.5-O6 | 23.53 |
| 1.5-O8 | 33.61 |
| 2.5-7 | 31.11 |
| 5.7 | 7.11 |
| 8.5 | 4.64 |

characterizes with the following phase transition sequence and temperatures: Cr<0° C.; SmA₁, 65.7° C.; N, 69.5° C.; Iso. The theoretical eutectic point is at the temperature of −11.35° C. At temperatures directly below the clearing point under a polarizing microscope a biphase system (SmA₁-N) and a triphase system (SmA₁-N-Iso) are observed.

Composition 4 (Five Components)

The composition comprising

| Acronym of compound | wt % |
|---|---|
| 1.5-O6 | 31.30 |
| 1.5-O8 | 43.00 |
| 5.7 | 10.26 |
| 5.9 | 8.40 |
| 8.5 | 7.04 |

characterizes with the following phase transition sequence and temperatures: Cr<0° C.; SmA₁, 58.5-64.5° C.; Iso. The theoretical eutectic point (−3.45° C.).

Composition 5 (Six Components)

The composition comprising

| Acronym of compound | wt % |
|---|---|
| 1.5-O6 | 22.33 |
| 1.5-O8 | 32.15 |
| 2.5-7 | 29.68 |
| 5.7 | 6.69 |
| 5.9 | 4.90 |
| 8.5 | 4.25 |

characterizes with following phase transition sequence and temperatures: Cr<0° C.; SmA₁, 65.5-78.0° C.; Iso. The theoretical eutectic point is at (−15.7° C.).

Composition 6 (Six Components)

The composition comprising

| Acronym of compound | wt % |
|---|---|
| 1.5-O6 | 20.52 |
| 1.5-O8 | 29.98 |
| 2.5-7 | 27.54 |
| 5.7 | 5.98 |
| 8.4 | 12.30 |
| 8.5 | 3.68 |

characterizes with the following phase transition sequence and temperatures: Cr<0° C.; SmA₁, 70.4-79.1° C.; Iso. The theoretical eutectic point is at (−21.2° C.).

Composition 7 (Seven Components)

The composition comprising

| Acronym of compound | wt % |
|---|---|
| 1.5-O6 | 18.86 |
| 1.5-O8 | 25.66 |
| 2.3-2 | 10.39 |
| 2.5-7 | 25.60 |
| 5.7 | 5.34 |
| 8.4 | 10.97 |
| 8.5 | 3.18 |

characterizes with the following phase transition sequence and temperatures: Cr<0° C.; SmA₁, 72.5; N, 80-83.2° C.; Iso. The theoretical eutectic point is at (−18.7° C.).

Composition 8 (Eight Components)

The composition comprising

| Acronym of compound | wt % |
|---|---|
| 1.5-O6 | 16.13 |
| 1.5-O8 | 24.59 |
| 2.3-2 | 9.05 |
| 2.5-7 | 22.28 |
| 5.7 | 4.34 |
| 8.4 | 9.32 |
| 8.5 | 2.47 |
| 12.5 | 11.82 |

characterizes with the following phase transition sequence and temperatures observed upon heating: Cr<0° C.; SmA, 80.0-89.4° C.; Iso and upon cooling: Iso 87.1-83.9° C. N, 82.0-76.2° C.; SmA₁ Cryst. <0° C. The theoretical eutectic point is at (−23.4° C.).

Composition 9 (Eight Components)

The composition comprising

| Acronym of compound | wt % |
|---|---|
| 1.5-O6 | 17.02 |
| 1.5-O8 | 25.72 |
| 2.3-2 | 9.49 |
| 2.5-7 | 23.37 |
| 5.7 | 4.66 |
| 8.4 | 9.85 |
| 8.5 | 2.71 |
| 11.5 | 7.18 |

characterizes with the following phase transition sequence and temperatures: Cr<0° C.; SmA₁, 75.4-79.0° C. N, 81.2-84.5° C.; Iso. The theoretical eutectic point is at (−21.82° C.).

Composition 10 (Eight Components)

The composition comprising

| Acronym of compounds | wt % |
|---|---|
| 1.5-O6 | 17.12 |
| 1.5-O8 | 25.86 |
| 2.3-2 | 9.54 |
| 2.5-7 | 23.51 |
| 5.7 | 4.69 |
| 8.4 | 9.91 |
| 8.5 | 2.72 |
| 11.5 | 6.65 |

characterizes with the following phase transition sequence and temperatures: Cr<0° C.; SmA₁, 75.3-78.5° C.; N, 87.1-90.8° C.; Iso. The theoretical eutectic point is at (−21.75° C.).

Composition 11 (Eight Components)

The composition comprising

| Acronym of compound | wt % |
|---|---|
| 1.5-O6 | 16.97 |
| 1.5-O8 | 25.64 |
| 2.3-2 | 9.46 |
| 2.5-7 | 23.29 |
| 5.7 | 4.64 |
| 8.4 | 9.85 |
| 8.5 | 2.69 |
| 9.3 | 7.46 |

characterizes with the following phase transition sequence and temperatures: Cr<0° C.; SmA₁, 73.9-83.9° C.; Iso. The theoretical eutectic point is at (−21.89° C.).

Composition 12 (Eight Components)

The composition comprising

| Acronym of compounds | wt % |
|---|---|
| 1.5-O6 | 17.60 |
| 1.5-O8 | 26.45 |
| 2.3-2 | 9.77 |
| 2.5-7 | 24.08 |
| 5.7 | 4.86 |
| 8.4 | 10.20 |
| 8.5 | 2.86 |
| 12.3 | 4.18 |

characterizes with the following phase transition sequence and temperatures: Cr<0° C.; SmA₁, 75.2-81° C.; N, 83.2-86.1° C.; Iso. The theoretical eutectic point is at (−20.91° C.).

### Example 8

Doped Composition.

Formulation of the composition comprising a pyrimidine or a four-ring fluorinated compound or a cyano compound.

Composition 13

The composition comprising

| | |
|---|---|
| Composition 6 | 78.45 wt % |
| Compound 19.7OCOO-9 | 21.55 wt % |

was formulated.

It has phase transitions: Cr<0° C.; SmA₁, 61.6-66.3° C.; N, 66.8-70.2° C.; Iso. The nematic phase is observed in a short temperature range below the clearing point in the comparison to composition 6, wherein it is not observed. The theoretical eutectic point is at (−21.92° C.).

Composition 14

The composition comprising

| | |
|---|---|
| Composition 7 | 80.35 wt % |
| Compound 19.7OCOO-9 | 19.65 wt % |

was formulated.

It has the phase transitions: Cr<0° C.; SmA₁, 61.2-63.7° C.; N, 70.7-73.6° C.; Iso. The theoretical eutectic point is at (−24.8° C.).

Composition 15

The composition comprising

| | |
|---|---|
| Composition 12 | 85.97 wt. % |
| Compound 19.4O-O8 | 14.03 wt. % |

was formulated.

It has phase transitions: Cr<0° C.; SmA, 66.2-69° C.; N, 72.5-75° C.; Iso. The theoretical eutectic point is at (−24° C.).

Composition 16

It was obtained composition comprising

| | |
|---|---|
| Composition 11 | 95.00 wt. % |
| Compound 16.5 | 5.00 wt. % |

It has the phase transitions: Cr<0° C.; SmA₁, 74.6-85.5° C.; Iso.

Composition 17

It was obtained composition comprising

| | |
|---|---|
| Composition 11 | 95 wt % |
| Compound 18.5 | 5 wt % |

It has phase transitions: Cr<0° C.; SmA₁, 83.6-96° C.; Iso.

Composition 18 (Eight Components)

The composition comprising

| Acronym of compounds | wt % |
|---|---|
| 1.5-O6 | 20.87 |
| 1.5-O8 | 30.54 |
| 5.7 | 6.07 |
| 8.4 | 12.19 |
| 8.5 | 3.72 |
| 9.3 | 9.46 |
| 10.5 | 8.05 |
| 18.5 | 9.10 |

characterized with the phase transitions: Cr<0° C.; SmA, 69-77.7° C.; Iso. The theoretical eutectic point is at (−15.97° C.). The suggested order and the position of molecules in the single smectic layer is shown in FIG. 1.

The alternate sequence of less polar alkyl, alcoxy compounds (1.5-O6 and 1.5-O8) and more polar terminally fluorinated compounds increases the stability of the SmA phase in the composition towards to the nematic phase of their components.

Composition 19 (Eight Components)

The composition comprising

| Acronym of compound | wt % |
|---|---|
| 1.5-O8 | 33.06 |
| 2.3-2 | 12.36 |
| 5.7 | 6.84 |
| 8.4 | 13.47 |
| 8.5 | 4.31 |
| 9.3 | 10.51 |
| 10.5 | 8.82 |
| 18.5 | 10.63 |

characterizes with the following phase transition sequence and temperatures: Cr<0° C.; SmA₁, 82.6-87.7° C.; Iso. The theoretical eutectic point is at (−13.24° C.).

Composition 20

It was obtained composition comprising

| | |
|---|---|
| Composition 19 | 85 wt % |
| Compound 19.4O-O8 | 15 wt % |

It has phase transitions: Cr<0° C.; SmA₁, 83.8-93° C.; Iso.

Composition 21 (Seven Components)

The composition comprising

| Acronym of compound | wt % |
|---|---|
| 1.5-O7 | 7.21 |
| 5.7 | 8.49 |
| 7.3 | 17.32 |
| 8.4 | 16.08 |
| 9.3 | 12.71 |
| 10.5 | 10.44 |
| 11.5 | 11.76 |
| 13.5 | 15.99 |

characterizes with following phase transition sequence and temperatures: Cr<0° C.; SmA, 104.5-111.7° C.; N, 127.3-130.8° C.; Iso. The theoretical eutectic point is at (−3.1° C.).

Composition 22 (Eight Components)

The composition comprising

| Acronym of compound | wt % |
|---|---|
| 1.5-O8 | 34.92 |
| 1.5-O7 | 05.39 |
| 5.5 | 10.76 |
| 5.7 | 07.34 |
| 5.5-CN | 05.62 |
| 8.4 | 14.30 |
| 9.3 | 11.21 |
| 11.5 | 10.46 |

characterizes with the following phase transition sequence and temperatures: Cr<0° C.; SmA₁78.8-84.5° C.; Iso. The theoretical eutectic point is at (−12.14° C.).

Composition 23

It was obtained composition comprising

| | |
|---|---|
| Composition 22 | 82.41 wt. % |
| Compound 19.4O-O8 | 17.59 wt. % |

It has the phase transitions: Cr<0° C.; SmA₁, 80.5-92.0° C.; Iso. The theoretical eutectic point is at (−17.56° C.).

Composition 24 (eight components)

The composition comprising

| Acronym of compound | wt % |
|---|---|
| 1.5-O7 | 05.33 |
| 5.5 | 10.76 |
| 5.7 | 07.32 |
| 5.5-CN | 05.60 |
| 8.4 | 14.30 |
| 9.3 | 11.19 |
| 11.5 | 10.45 |
| 6.5 | 35.05 |

characterizes with the following phase transition sequence and temperatures: Cr<0° C.; SmA₁, 93-98.2° C.; N, 106.5-107° C.; Iso. The theoretical eutectic point is at (−12.6° C.).

Composition 25

It was obtained composition comprising

| | |
|---|---|
| Composition 24 | 97.00 wt % |
| Compound 4.5-CN | 3.00 wt % |

It has the phase transitions: Cr<0° C.; SmA, 88.0° C.; then the triphase system SmA-N-Iso is observed up to the temperature of 109.3° C.

### Example 9

The Composition Doped with Ionic Compounds and its Electrooptical Properties.

The smectic composition doped with the ionic compound II.16b—the estimation of optimum concentration of the dopant.

The optimum concentration of the ionic dopant was recognized on the example of compound II.16b [C₁₆H₃₃DBU]⁺Br. The smectic A composition 18 was doped in the amounts: 0.05, 0.1, 0.3, 0.4 and 0.5 wt % in the following way: 100 mg of the smectic composition was weighted, 0.1 cm³ of fluorobenzene was added and combined with 5 μl or 11 μl or 32 μl or 43 μl or 54 μl of the solution of the ionic compound dissolved in fluorobenzene with concentration 0.0204 mol/dcm³. The mentioned amounts ensure the desired concentrations of the ionic compound of 0.05 or 0.1 or 0.3 or 0.4 or 0.5 wt. % in the smectic A composition.

The components were carefully mixed and then heated to temperature of 90° C. to evaporate the solvent. Then heating was continued under lower pressure (at 0.2 mmHg) for the total removal of the solvent and the composition was carefully mixed by shaking it at the isotropic state.

Then the cell with the gap of 15 μm and the dimension of electrodes 12.7 mm×12.7 mm was filled with the mentioned composition being in the isotropic state. Its intensity-voltage characteristics (FIG. 2) were measured for the scattering state and the clear state. The obtained results are listed in Table 6.

TABLE 3
Conductivity σ, threshold voltage Vₜₕ(sc), saturation voltage Vₛ(sc) for the scattering state
and threshold voltage Vₜₕ(c), saturation voltage Vₛ(c) for the clear (erase) state of doped composition
18 with ionic compound 12.16b at different frequencies of driving electric field at RT.

| | Concentration | Conductivity | Vₜₕ(sc) [V] | Vₜₕ(c) [V] | | | |
|---|---|---|---|---|---|---|---|
| | of dopant | σ(Ohm⁻¹cm⁻¹) | Vₛ(sc) [V] | Vₛ(c) [V] | | | |
| Sample | wt. % | f = 100 Hz | f = 50 Hz | f = 0.8 kHz | f = 1 kHz | f = 3 kHz | f = 5 kHz |
| 1 | 0.05 | 5.1 · 10⁻¹⁰ | 180 | 100 | 100 | 100 | 90 |
| | | | 270 | 220 | 220 | 170 | 110 |
| 2 | 0.1 | 6.1 · 10⁻¹⁰ | 220 | 110 | 110 | 120 | 140 |
| | | | 250 | 130 | 140 | 140 | 140 |
| 3 | 0.3 | 7 · 10⁻¹⁰ | 160 | 110 | 120 | 140 | 140 |
| | | | 170 | 130 | 130 | | 150 |
| 4 | 0.4 | 1.30 · 10⁻⁹ | 100 | 130 | 140 | 140 | 170 |
| | | | 170 | 180 | 150 | 160 | — |
| 5 | 0.5 | 1.0 · 10⁻⁹ | 100 | 110 | 120 | 110 | 110 |
| | | | 150 | 150 | 150 | 150 | 150 |

The DS effect was observed for all concentrations of ionic dopant II.16b. With the increase of dopant concentration the threshold voltage and the saturation voltage decrease. The clearing effect (erasing of scattering) was observed for all concentrations also. The threshold voltage Vₜₕ(c) is changing in a small degree, when the concentration of the dopant is changing; the saturation voltage Vₛ(c) is decreasing and then is increasing with the increase of the dopant concentration. For the small concentration of the dopant the saturation voltage depends strongly on the frequency of the driving electric field. At the concentration of 0.5 wt. % a strong heating of the cell was observed. The conductivity grows proportionally to the concentration of the ionic dopant.

The concentration of the ionic dopant in the range of 0.3-0.4 wt % ensures an optimum feature for the scattering state and the clear state.

The observed level of conductivity in the doped liquid crystal smectic composition is hundred time lower than that observed in the isotropic liquid (fluorobenzene).

The layer structure of the liquid crystal smectic A₁ composition decreases the mobility of ions in electric field in comparison to the observed one in the isotropic phase of fluorobenzene.

### Example 10

The Comparison of the Properties of the Compositions Doped with Different Ionic Compounds.

The composition 18 was doped always the same mole amount of a ionic dopant equivalent to 0.4 wt % of compound [C₁₆H₃₃DBU]⁺Br⁻, that is 8.78·10⁻³ mol/kg.

In U.S. Pat. No. 7,628,292B1 the used concentration of the ionic compound was 6.66·10⁻³ mol/kg—near to used one here. The obtained experimental results are listed in Table 4.

TABLE 4
The threshold voltage Vₜₕ(sc) and the saturtion voltage Vₛ(sc) for the scattering state and Vₜₕ(c),
Vₛ(c) for the clear state, respectively, at the different frequencies of the driving electric field
and conductivity σ at 100 Hz measured for doped composition 18 (prepared in fluorobenzene) at RT.

| | | Vₜₕ(sc) [V] | Vₜₕ(c) [V] | | | | | |
|---|---|---|---|---|---|---|---|---|
| Ionic | concentration | Vₛ(sc) [V] | Vₛ(c) [V] | | | | σ | |
| compound | mol/kg | f = 50 Hz | f = 0.8 kHz | f = 1 kHz | f = 3 kHz | f = 5 kHz | [Ohm⁻¹cm⁻¹] | Quality |
| I.16b | 8.78 · 10⁻³ | no dynamic scattering | | | | | 5.7 · 10⁻¹¹ | |
| I.1g12 | 8.78 · 10⁻³ | no dynamic scattering | | | | | 1.55 · 10⁻¹¹ | |
| II.2b | 6.58 · 10⁻³ | 300 | 120 | | | | 3.0 · 10⁻⁹ | W |
| | | | 120 | | | | | |
| II.16b | 8.78 · 10⁻³ | 100 | 130 | 140 | 140 | 170 | 1.3 · 10⁻⁹ | G |
| | | 170 | 180 | 150 | 160 | | | |
| II.16b* | 8.78 · 10⁻³ | 100 | 100 | 150 | 130 | 150 | 1.2 · 10⁻⁹ | G |
| | | 230 | 150 | 170 | 170 | 170 | | |
| II.16c | 8.78 · 10⁻³ | 190 | | 120 | | | 4.6 · 10⁻¹⁰ | W |
| | | 250 | | — | | | | |
| II.16d | 6.58 · 10⁻³ | 90 | 110 | 110 | 90 | | 6.84 · 10⁻¹⁰ | W |
| | | 170 | 140 | 130 | 120 | | | |
| II.16k | 8.78 · 10⁻³ | 120 | | 90 | 90 | 90 | 3.9 · 10⁻⁹ | G |
| | | 220 | | 120 | 120 | 130 | | |
| II.16n | 8.78 · 10⁻³ | 220 | | | | | 1.8 · 10⁻⁸ | W |
| | | 270 | | | | | | |
| II.16g12 | 8.78 · 10⁻³ | 100 | 140 | 140 | 140 | 150 | 1.1 · 10⁻⁹ | G |
| | | 190 | 150 | 150 | 150 | | | |
| III.3b | 6.58 · 10⁻³ | 120 | 80 | | | | 1.1 · 10⁻⁹ | W |
| | | 270 | 100 | | | | | |
| IIIk | 6.58 · 10⁻³ | 120 | 100 | 110 | 100 | 110 | 2.7 · 10⁻⁹ | G |
| | | 200 | 130 | 120 | 110 | 120 | | |
| IVb | 6.58 · 10⁻³ | 120 | 150 | | | | 2.2 · 10⁻¹⁰ | W |
| | | 330 | 150 | | | | | |
| IVc | 8.78 · 10⁻³ | 100 | 130 | 130 | | | 1.5 · 10⁻¹⁰ | W |
| | | — | | | | | | |
| IVk | 8.78 · 10⁻³ | 110 | 100 | 100 | 100 | 140 | 4.1 · 10⁻⁹ | M |
| | | 230 | 150 | 150 | 140 | | | |
| IVn | 8.78 · 10⁻³ | 90 | 100 | 130 | 130 | 130 | 8.1 · 10⁻⁹ | M |
| | | 240 | 140 | | | | | |
| IVn | 2.19 · 10⁻³ | 200 | 100 | 100 | 90 | 90 | 2.19 · 10⁻⁹ | M |
| | | 220 | 140 | 140 | 120 | 110 | | |
| IVh8 | 8.78 · 10⁻³ | 130 | | | | | 1.4 · 10⁻¹⁰ | W |
| IVg12 | 8.78 · 10⁻³ | 190 | 90 | 90 | 80 | 90 | 2.7 · 10⁻¹⁰ | W |
| | | 320 | 100 | 90 | 100 | 90 | | |
| Ve12 | 6.58 · 10⁻³ | 260 | 70 | | | | 3.7 · 10⁻¹⁰ | W |
| | | 280 | 80 | | | | | |

*chloroform as solvent
G—parameters of the dynamic state and the clear state - good
M—parameters of the dynamic state and the clear state - moderate
W—parameters of the dynamic state and the clear state - weak

Ionic dopants: amidynium salts and crown ether complexes characterized by specific conductivity in range 10⁻¹⁰-10⁻⁸ Ohm⁻¹cm⁻¹. The amidynium salts have a little higher conductivity than the complexes of the crown ethers in the composition 18. The best parameters of scattering state and the clear state were observed for the composition doped with II.16b, II.16k and II.16g12. Those ionic compounds ensure the conductivity in the range of 1.1-1.3·10⁻⁹ Ohm⁻¹cm⁻¹. When the anion was ClO₄ (compound II.16c) or phenolate (compound II.16n) the significantly higher threshold voltage was observed for the scattering state and the quality of the clear state was not good.

The composition 18 doped with complex of crown ether exhibits a worse quality of the scattering as well the clear state except the compound III.k, which ensures the similar high level of conductivity 2.7·10⁻⁹ Ohm⁻¹cm⁻¹ and the observed parameters of the scattering state as well as the clear state are the same good as for amidynium salts mentioned above.

For comparison—the observed level of conductivity in the presence of hexadecyltrimethyl ammonium bromide (compound I.16b) and tetramethylammonium dodecylbenzenesulphonate (compound I.1g12) was very low below 5.0·10⁻¹¹ Ohm⁻¹cm⁻¹. Here the effect of dynamic scattering was not observed up to voltage 400V.

Ammonium ionic compounds are not useful as ionic dopants to the smectic A₁ composition according to the present disclosure, because their solubility and specially their conductivity observed are too low, although in case of compositions composed of cyanocompounds were good.

### Example 11

The Composition 18 Doped Simultaneously with Two Kind of Ionic Compounds.

In Table 5 the properties of mixture 18 doped simultaneously two ionic compounds are compared. They contain the same total amount of 8.78·10⁻³ mol/kg of the ionic dopant.

TABLE 5
The threshold voltages and the saturation voltages for the scattering state
Vₜₕ(sc), Vₛ(sc) and the clear state Vₜₕ(c), Vₛ(c), respectively, and specific
conductivity σ in composition 18 comprising two kinds of ionic dopants at RT.

| | | Vₜₕ(sc) [V] | Vₜₕ(c) [V] | | | σ, | |
|---|---|---|---|---|---|---|---|
| Ionic dopant 1 | Ionic dopant 2 | Vₛ(sc) [V] | Vₛ(c) [V] | | | [Ohm⁻¹cm⁻¹] | |
| [mol/kg] | [mol/kg] | f = 50 Hz | f = 0.8 kHz | f = 1 kHz | f = 3 kHz | f = 100 Hz | Quality |
| (C₄H₉)₄N⁺CO⁻₄ | II.16b | 90 | 110 | 140 | | 9.3 · 10⁻¹⁰ | W |
| 6.58 · 10⁻³ | 2.2 · 10⁻³ | 220 | 170 | | | | |
| II.16n | II.16b | 70 | 120 | | | 32 · 10⁻⁸ | W |
| 6.58 · 10⁻³ | 2.2 · 10⁻³ | | | | | | |
| II.16b | | 120 | 120 | 150 | 130 | 1.2 · 10⁻⁹ | G |
| 8.78 · 10⁻³ | | 220 | 150 | 160 | 170 | | |
| II.16g12 | II.16b | 70 | 120 | | | 5.7 · 10⁻¹⁰ | G |
| 6.58 · 10⁻³ | 2.2 · 10⁻³ | 120 | 180 | | | | |
| II.16b | IVg12 | 100 | 120 | 150 | 160 | 9.3 · 10⁻¹⁰ | G |
| 2.2 · 10⁻³ | 6.58 · 10−3 | 210 | 140 | 150 | | | |
| II.16b | IV.g12 | 100 | 120 | 120 | 120 | 1.26 · 10⁻⁹ | G |
| 3.28 · 10⁻³ | 3.28 · 10⁻³ | 230 | 130 | 130 | 140 | | |

G—parameters of the dynamic state and the clear state - good
W—parameters of the dynamic state and the clear state - weak

The composition 18 doped with the ionic compound II.16b (1-hexadecyl-1,8-diazabicyclo[5,4,0]undec-7-enium bromide) and with II.16g12 (1-hexadecyl-1,8-diazabicyclo[5,4,0]undec-7-enium 4-dodecylbenzenesulphonate) exhibits the low threshold voltage and saturation voltage for the scattering state (70 and 120V, respectively). The threshold voltage and the saturation voltage for clear state are higher (120 and 180V, respectively).

Composition 18 doped with the ionic compound II.16b and with the compound IV.g12 (complex dibenzo-18-crown-6 ether with potassium 4 dodecylbenzenesulphonate) also shows the excellent parameters of the scattering state (100V and 210V) and the clear state (120V and 140V at 0.8 Hz) in the presence of an excess of crown ether complex or as well as when the both ionic compounds are in the equimolar amounts.

The presence of two kind of dopants causes that the electrooptic switching curve (light transmission versus voltage) is more steep.

### Example 12

In Table 6 the properties of the composition 21 doped with an amidinium salt or potassium complex of a crown ether are compared. The composition 21 has the phase sequence Cr-SmA₁-N-Iso and the higher value of dielectric anisotropy than composition 18.

TABLE 6
The threshold voltages and the saturation voltages for the scattering state Vₜₕ(sc), Vₛ(sc) and the clear
state Vₜₕ(c), Vₛ(c), respectively, and the value of specific conductivity σ in doped composition 21 at RT.

| | | Vₜₕ(sc) [V] | Vₜₕ(c) [V] | | | | | σ, | |
|---|---|---|---|---|---|---|---|---|---|
| Ionic | Concentration | Vₛ(sc) [V] | Vₛ(c) [V] | | | | | [Ohm⁻¹cm⁻¹] | |
| dopant | mol/kg | f = 50 Hz | f = 0.8 kHz | f = 1 kHz | f = 3 kHz | f = 5 kHz | f = 7 kHz | f = 100 Hz | Quality |
| II.16b | 6.58 · 10⁻³ | 60 | 80 | 90 | 70 | 70 | 70 | 3.24 · 10⁻⁹ | G |
| | | 120 | 90 | 100 | 90 | 80 | 80 | | |
| II.16g12 | 6.58 · 10⁻³ | 120 | 70 | 70 | 70 | 70 | 70 | 7.42 · 10⁻¹⁰ | G |
| | | 130 | 70 | 70 | 70 | 70 | 80 | | |
| IVg12 | 6.58 · 10⁻³ | 130 | 80 | 50 | 70 | 60 | 60 | 4.68 · 10⁻¹⁰ | G |
| | | 170 | 80 | 50 | 70 | 60 | 60 | | |

G—parameters of the dynamic state and the clear state - good
M—parameters of the dynamic state and the clear state - moderate
W—parameters of the dynamic state and the clear state - weak

The lower threshold voltages and the saturation voltages are observed for the composition 21 than for the composition 18.

The presence of ionic dopant II.16b in the composition 21 leads to the very low threshold voltage for the scattering state.

The presence of ionic dopants II.16g12 or IVg12 leads to the higher threshold voltages, but the electrooptic curves are steeper. The threshold voltages for the clear state are low for all three dopants. It equals only 50V at 1 kHz for IVg12. The electrooptical curves are very steep—the saturation voltage of the clear state is only a little higher than the threshold voltage.

### Example 13

Composition with a Radical Stabiliser.

0.1 wt % of the ionic dopant II.16b or III.g12 and 0.3 wt % of the stabilizer of the formula 29, wherein R9 is tert-butyl CH(CH₃)₃ and R8 is sec-butyl CH(CH₃)C₂H₅ was added to the composition 21.

The stable and good quality scattering state as well as the clear state was obtained with the parameters given below:

TABLE 7
The threshold voltages and the saturation voltages for the scattering state Vₜₕ(sc),
Vₛ(sc) and the clear state Vₜₕ(c), Vₛ(c), respectively, and the value of specific
conductivity σ in composition 21 containing stabilizer of the formula 29 at RT.

| | Vₜₕ(sc) [V] | Vₜₕ(c) [V] | | | | | σ |
|---|---|---|---|---|---|---|---|
| Ionic | Vₛ(sc) [V] | Vₛ(c) [V] | | | | | [Ohm⁻¹cm⁻¹] |
| dopant | f = 50 Hz | f = 0.8 kHz | f = 1 kHz | f = 3 kHz | f = 5 kHz | f = 7 kHz | f = 100 Hz |
| II.16b | 80 | 70 | 70 | 70 | 70 | 70 | 2.0 · 10⁻⁹ |
| | 110 | 70 | 70 | 80 | 80 | 80 | |
| III.g12 | 70 | | | 70 | 70 | 70 | |
| | 110 | | | 80 | 80 | 80 | 7.0 · 10⁻⁹ |

The presence of the radical stabilizer causes the decrease of the needed amount of ionic dopant to involve the scattering state.

The presence of the stabilizer ensures good parameters of the scattering state as well as the clear state for the low concentration of ionic dopant II.16b only 0.1 wt %. The clear state is observed at 0.8 kHz yet.

For the ionic dopant III.g12 the observed threshold voltages and saturation voltages are similar to the observed ones for II.16b, but the clear state is observed at higher frequency 3 kHz).

### Example 14

Composition with a Dye.

0.1 wt % of ionic dopant II.16b and 0.3 wt % of the ionic dye with formula 34 was added to the composition 21.

The stable and good quality of the scattering state as well as the clear state was obtained with a low concentration of the ionic dopant. Their parameters are given in Table 8.

TABLE 8
The threshold voltages and the saturation voltages for the scattering state Vₜₕ(sc),
Vₛ(sc) and the clear state Vₜₕ(c), Vₛ(c), respectively, and the value of specific
conductivity σ in composition 21 containing ionic dye of the formula 34 at RT.

| | Vₜₕ(sc) [V] | Vₜₕ(c) [V] | | | | | σ | |
|---|---|---|---|---|---|---|---|---|
| Ionic | Vₛ(sc) [V] | Vₛ(c) [V] | | | | | [Ohm⁻¹cm⁻¹] | |
| dopant | f = 50 Hz | f = 0.8 kHz | f = 1 kHz | f = 3 kHz | f = 5 kHz | f = 7 kHz | f = 100 Hz | Quality |
| II.16b | 130 | 70 | 70 | 70 | 70 | 70 | 5.58 · 10⁻⁹ | G |
| | 170 | 80 | 80 | 80 | 90 | 90 | | |

For these concentration of the ionic dye the composition is still nearly colourless.

### Example 15

0.3 wt % of the ionic dopant II.16b and 2 wt % of blue color dichroic dye (AB4, producer—Nematel) was added to the composition 21. The stable and good quality of the scattering state (with dark blue colour) as well as of the clear state (light blue colour) was obtained with the parameters given in Table 9.

TABLE 9
The threshold voltages and the saturation voltages for the scattering state Vₜₕ(sc), Vₛ(sc) and the clear state Vₜₕ(c),
Vₛ(c), respectively, and the value of specific conductivity σ in composition 21 containing blue color dichroic dye AB4 at RT.

| | | Vₜₕ(sc) [V] | Vₜₕ(c) [V] | | | | | | |
|---|---|---|---|---|---|---|---|---|---|
| Ionic | Concentration | Vₛ(sc) [V] | Vₛ(c) [V] | | | | | σ, | |
| dopant | mol/kg | f = 50 Hz | f = 0.8 kHz | f = 1 kHz | f = 3 kHz | f = 5 kHz | f = 7 kHz | [Ohm⁻¹cm⁻¹] | Quality |
| II.16b | 6.58 · 10⁻³ | 80 | 60 | 70 | 60 | 70 | 70 | 3.1 · 10⁻⁸ | G |
| | | 120 | 70 | 80 | 70 | 80 | 80 | | |

The presence of the dye increases the conductivity more than 10 times, the other electrooptic parameters are similar as for the composition without the dye.

### Example 16

Preparation of the Composition in a Large Scale.

100 g of the composition 18 and 100 cm³ of chloroform (CHCl₃) was placed in a conic flask and then the solution prepared from 0.3 g of the ionic compound II.16b and 50 cm³ of chloroform was added. The mixture was heated and shaken in nitrogen at 60° C. until the homogenous solution was obtained. The solution was filtered through a micropore membrane (Millipore with diameter of pore 0.22 μm) under low pressure. Then the solution was placed in the flask of a rotation evaporator and the solvent was removed at lower pressure. Then through the system the dry nitrogen was flowing for 2 hours at the temperature of 80° C. and the pressure of 1 mmHg. The system was cooled to RT and the flask was closed and kept in a chamber filled with the dry nitrogen.

When other inert gas was more preferable, argon or crypton was flowed through the system.

### Example 17

Manufacturing Device (Display) and Measurement of its Electrooptical Parameters.

A display for the test was made as the cell consists of two glass substrates. Float typed glass with indium-tin-oxide (ITO) layer of specific resistivity 100 Ohm/sq was used, which is transparent in the visible range of light.

ITO electrodes with active surface of 2 cm×2 cm were made by wet etching. Then they were washed with deionic water of specific resistivity of 18 MOhm at the presence of ultrasonic agitation and subsequently the substrates were dried at temperature of 430 K to remove a residual water. Then on each electrode on the glass substrates a thin (c.a. 60 nm) layer of polyimide RN1112 was deposited by a spincoating method. Then the substrates with electrodes and the polyimide layer were dried at about 70° C. for removing the solvent and were cured at the temperature of 180° C. Then one of the electrodes was covered with glass spacers in form of microrods of a diameter of 5-20 μm, preferably 15 μm, in amounts of 1 piece/mm² using their dispersion in ethanol sprayed under IR heating source.

On the second electrode, a line of thermocurred glue line was deposited by a serigraphy method. In the glue line a two gaps were formed for cell filling with the liquid crystal composition and for the air evacuation. Such a prepared substrates were assembled and were pressed to obtain the flat-parallel gap of diameter equal to the diameter of spacers used. The assembled cell was heated at 180° C. for currying of the glue. Then the cell was put on a metal heater having temperature above the clearing point of the liquid crystal composition.

Then, the cell gap was filled with the liquid crystal composition by a capillary action. The cell with the liquid crystal composition was slowly cooling (0.5° C./min) to the RT.

Cells electrodes were wired using a low melting alloy and an ultrasonic soldering system. The uniform homeotropic orientation of the optic axis of the smectic A structure in the cell gap was tested by using of a polarizing microscopic method.

1. A smectic A composition with the positive dielectric anisotropy exhibiting a monolayer smectic A structure (SmA₁), which shows a phase transition from a smectic A phase to an isotropic phase (SmA-Iso) or a phase transition from a smectic A phase through a nematic phase to an isotropic phase (SmA-N-Iso) and comprises at least two fluorinated compounds selected from the families of fluorinated derivatives of biphenyls and terphenyls expressed by the general formulae 1-12, 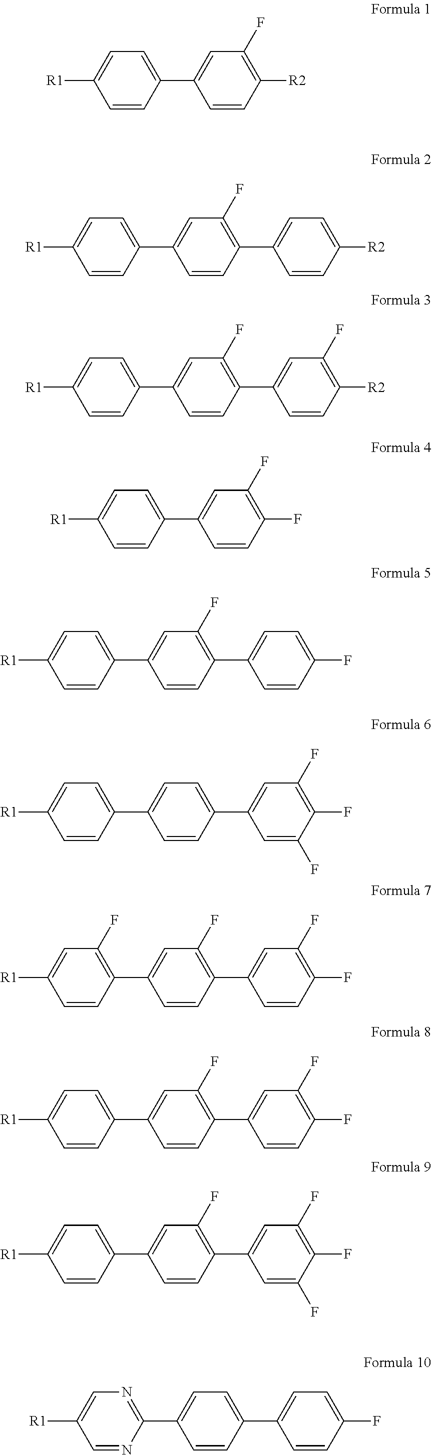 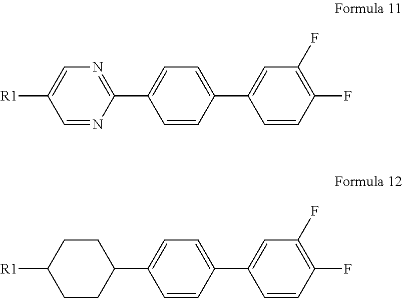
wherein
the terminal chain R1 is an alkyl group (H₂ₙ₊₁Cₙ) containing from 1 to 15 carbon atoms (n=1-15),
the terminal chain R2 is an alkyl group (CₘH₂ₘ₊₁) or an alcoxy group (OCₘH₂ₘ₊₁) or an alkyl carbonato group (OCOOCₘH₂ₘ₊₁), each containing independently from 1 to 15 carbon atoms (m=1-15)
and wherein at least one of the said fluorinated compounds is a compound expressed by the general formulae 1 or 2 or 3;

2. A smectic A composition according to claim 1, which contains additionally
at least one four-ring fluorinated compound preferably selected from the compounds expressed by the general formulae 13-18: 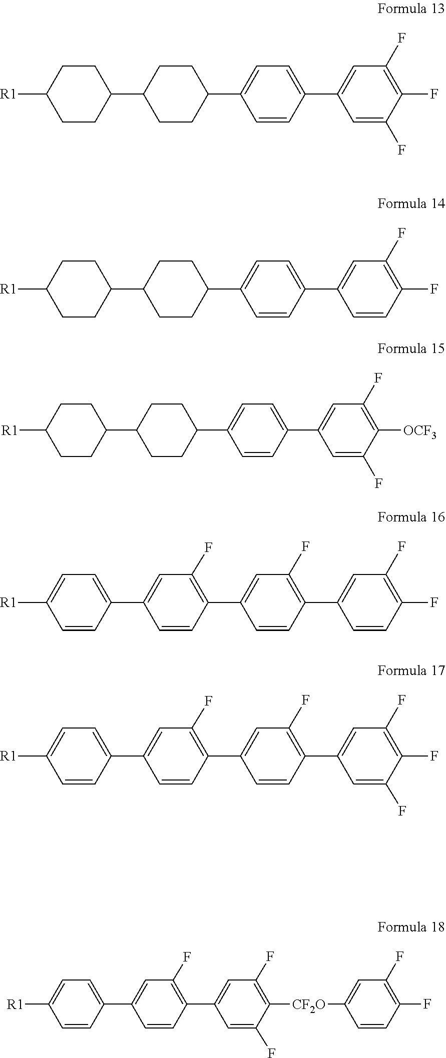
or at least one derivative of pyrimidine expressed by the general formulae 19-20 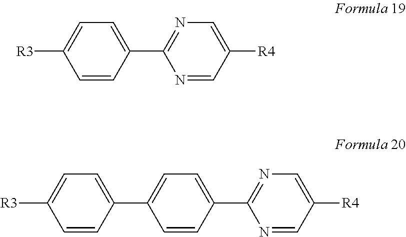
or a derivative of terphenyl of the general formula 21 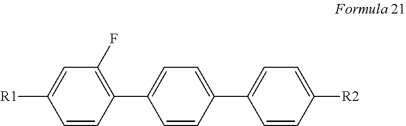
or two or all said compounds,
wherein terminal group R1 is an alkyl group (H₂ₙ₊₁Cₙ) containing from 1 to 15 carbon atoms (n=1-15),
R3 and R4 are independently an alkyl or an alkoxy or an alkyl carbonato group, each containing from 1 to 15 carbon atoms.

3. A smectic A composition according to claim 1, which contains additionally at least one liquid crystalline cyanocompound preferably selected from compounds expressed by the general formulae 4-12, wherein the terminal fluorine atom (F) is replaced by the cyano group (CN) and its concentration is below 25 wt. %, preferably in range of 3 wt. % to 20 wt. %.

4. A smectic A composition according to claim 1, which contains additionally at least one or more ionic dopants with delocalized charge of cation selected from ionic compounds expressed by the general formulae 22-26, 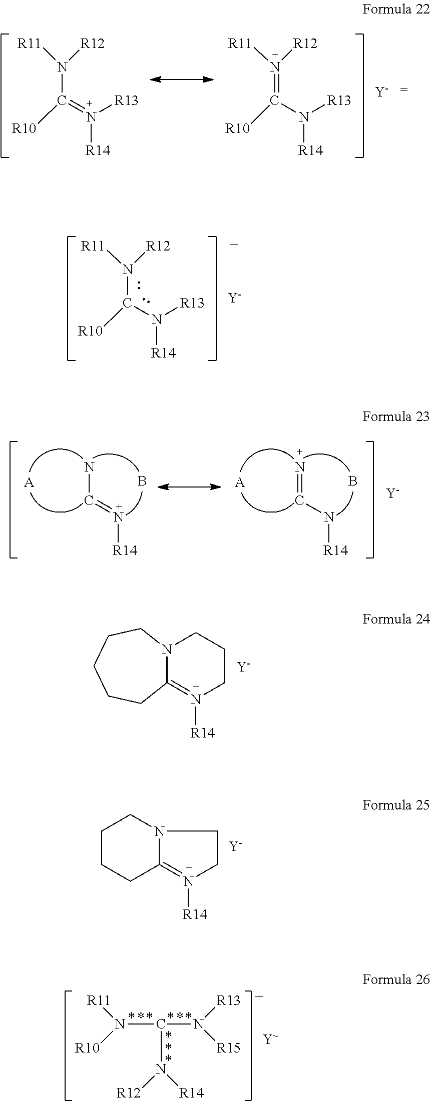
wherein the substituents R10, R11, R13 and R15 in the cationic part of the molecule are independently a hydrogen atom or an alkyl group or an alkylcyclohexyl group or a phenyl group or an alkylphenyl group and they are the same or different and contain 1 to 25 carbon atoms;
the substituents R12 and R14 are an alkyl group containing independently from 1 to 25 carbon atoms, preferably from 12 to 18 carbon atoms;
in the formula 23 symbols A and B denote a part of the ring containing from two to five methylene groups leading to specially preferable compounds with the system of rings expressed by formulae 24 (A=5, B=3) or 25 (A=4, B=2), wherein R14 is preferably an alkyl group containing from 2 to 18 carbon atoms;
the anion Y⁻ denotes an organic or an inorganic anion preferably: Cl^{θ}— (a), Br^{θ}— (b), ClO₄^{θ}— (c), CF₃SO₃^{θ}— (d), R5-SO₃^{θ}— (e), SCN^{θ}— (f), 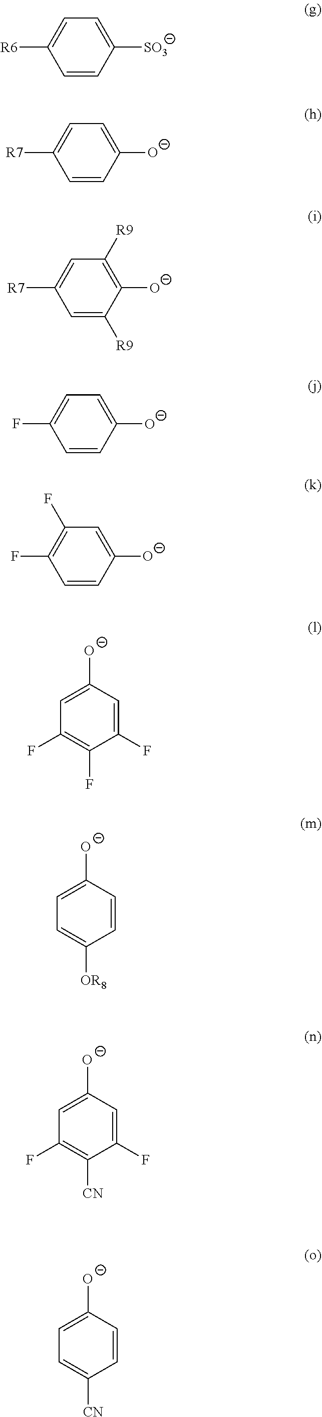 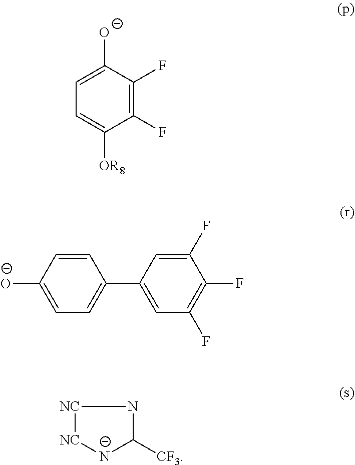
wherein substituents R5, R6, R7 and R8 are meaning a hydrogen or an normal alkyl group or an iso-alkyl group or a sec-alkyl group each containing independently from 1 to 20 carbon atoms and R9 is a branched alkyl group preferably the tert-butyl group.

5. A smectic A composition according to claim 1, which contains additionally at least one or more ionic dopants with a delocalized charge of cation selected from ionic compounds being complexes formed from the salts of alkaline metals preferably from potassium salts and crown ethers preferably expressed by the general formulae 27 and 28, 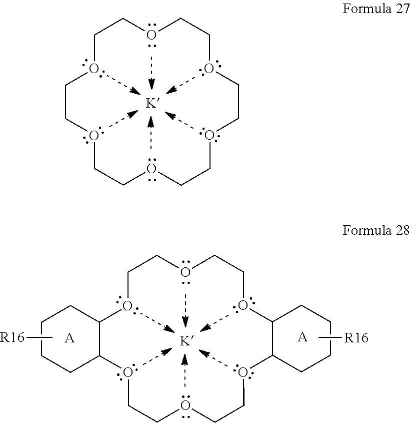
wherein
symbol A is meaning a cyclohexane ring or a benzene ring,
substituents R16 are independently a hydrogen atom or an alkyl group the same or different containing each from 1 to 16 carbon atoms;
anion Y⁻ is an organic anion or an inorganic anion preferably: Cl^{θ}— (a), Br^{θ}— (b), ClO₄^{θ— (c), CF}₃SO₃^{θ}— (d), R5-SO₃^{θ}— (e), SCN^{θ} (f) 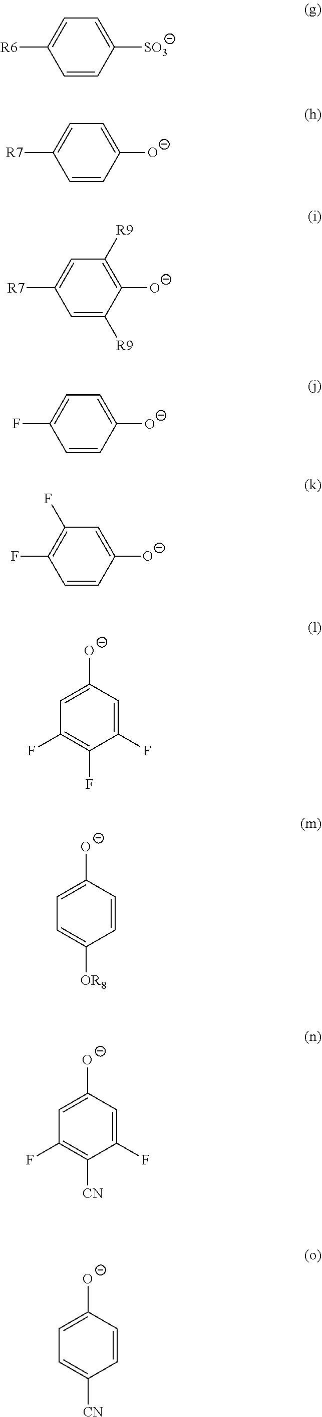 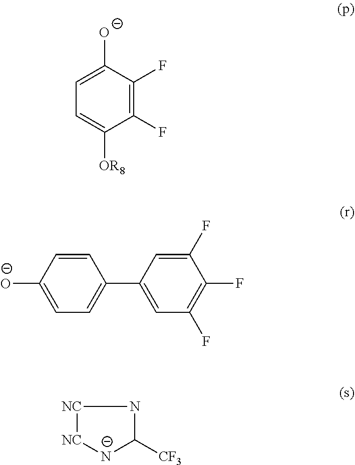
R5, R6, R7 and R8 are meaning a hydrogen or an normal alkyl group or an iso-alkyl group or a sec-alkyl group each containing independently from 1 to 20 carbon atoms and R9 is a branched alkyl group preferably the tert-butyl group

6. A smectic A composition according to claim 1, which contains additionally simultaneously two kinds of ionic dopants.

7. A smectic A composition according to claim 4 into which a dye is added or a few dyes are added with isotropic or anisotropic absorption of light with preferably of formula 34 or 35 or an nonionic structure preferable an anthraquinone dye in the amount below of 3 wt. %, preferably in range 0.5 wt % to 1.5 wt. %.

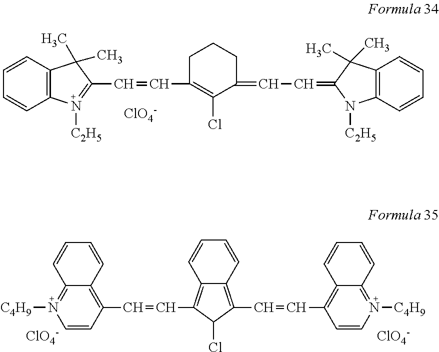

8. An ionic compound expressed by the general formulae 22-26, 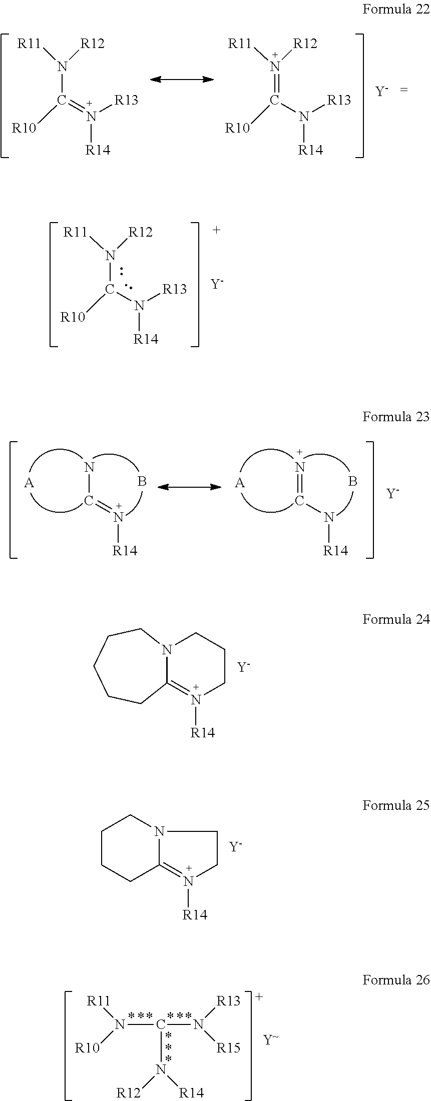
wherein
substituents R10, R11, R13 and R15 in cationic part of the molecule are meaning independently a hydrogen atom or an alkyl group or an alkyl cyclohexyl group or a phenyl group or an alkyl phenyl group and they are the same or different and contain 1 to 25 carbon atoms; the substituents R12 and R14 are an alkyl group containing independently from 1 to 25 carbon atoms, preferably from 12 to 18 carbon atoms;
in the formula 23 symbols A and B denote a part of the ring containing from two to five methylene group leading to specially preferable compounds with the system of rings expressed by formulae 24 (A=5, B=3) or 25 (A=4, B=2), wherein R14 is preferably an alkyl group containing from 2 to 18 carbon atoms;
the anion Y⁻ denotes an organic or an inorganic anions preferably: Cl^{θ}— (a), Br^{θ}— (b), ClO₄^{θ}— (c), CF₃SO₃^{θ}— (d), R5-SO₃^{θ}— (e), SCN^{θ} (f) 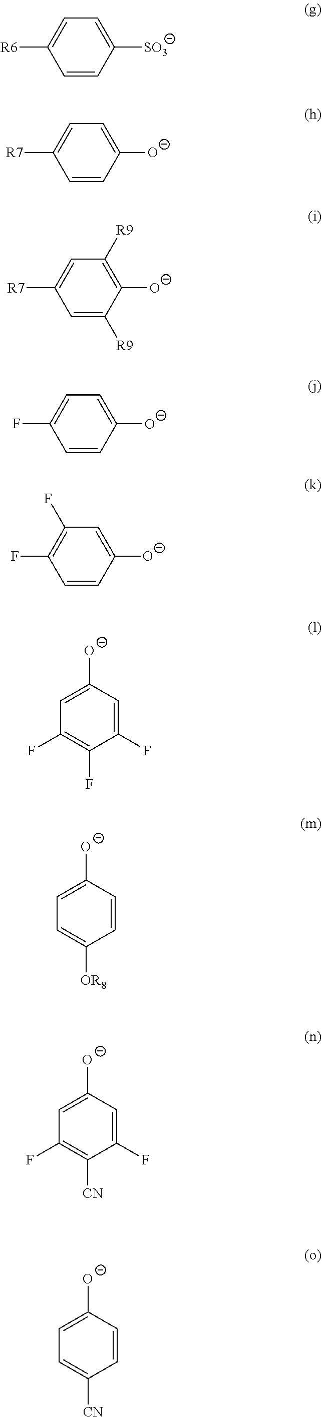 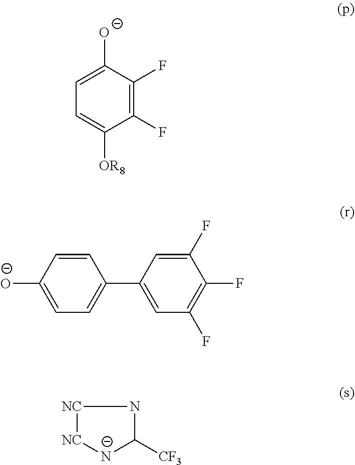
wherein substituents R5, R6, R7 and R8 denote a hydrogen or an normal alkyl group or an iso-alkyl group or a sec-alkyl group each containing independently from 1 to 20 carbon atoms, R9 is a branched alkyl group preferably the tert-butyl group used as an ionic dopant to increase conductivity in smectic A compositions.

9. An ionic compound expressed by the general formulae 27 and 28, 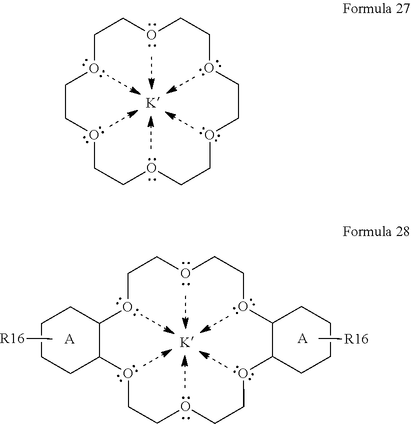
wherein
symbol A is a cyclohexane ring or a benzene ring,
substituents R16 are independently a hydrogen atom or an alkyl group the same or different containing each from 1 to 16 carbon atoms;
anion Y⁻ is an organic anion or an inorganic anion preferably: Cl^{θ}— (a), Br^{θ}— (b), ClO₄^{θ}— (c), CF₃SO₃^{θ}— (d), R5-SO₃^{θ}— (e), SCN^{θ} (f) 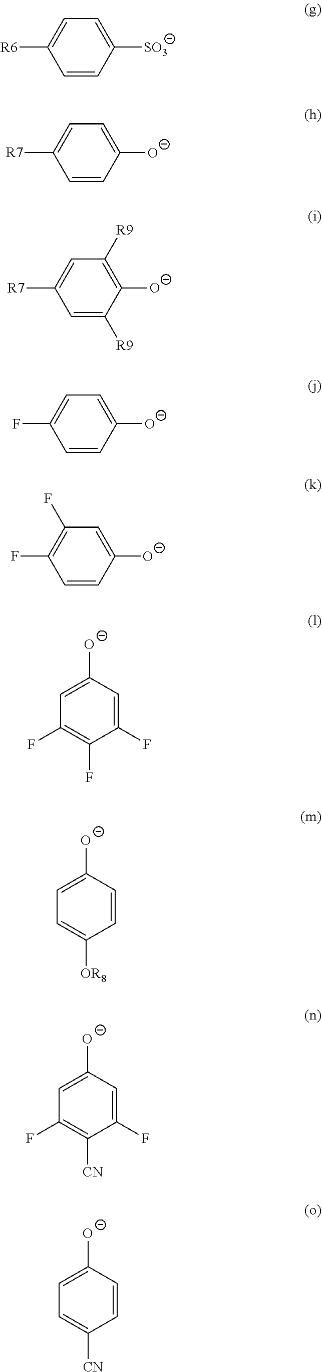 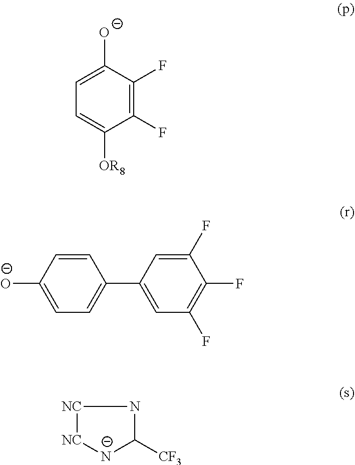
used as an ionic dopant to increase conductivity in smectic A compositions.

10. A method of obtaining of a smectic A composition doped with the ionic compound according to claim 4 wherein the composition is dissolved in an organic solvent preferably in fluorobenzene or chloroform and an ionic compound is dissolved in an organic solvent preferably fluorobenzene or chloroform, then both solutions are combined, then mixed, and filtered through micropore membrane; the solvent is evaporated at first under normal pressure then at lower pressure, the rest is heated under flow of an inert gas; the ionic dopant is added in such amount to obtain conductivity of order of 10⁻⁹ Ohm⁻¹cm⁻¹ or higher.

11. A method of obtaining of smectic A composition according to claim 10 wherein to the solution containing liquid crystal composition a compound is added additionally with ability to hinder radical reaction from a phenol family expressed by the formula 29, 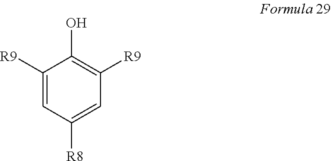
wherein substituent R9 in orto-position to hydroxy group is a bulk substituent preferably tert-butyl group or an amine derivative expressed by formulae 30-33, 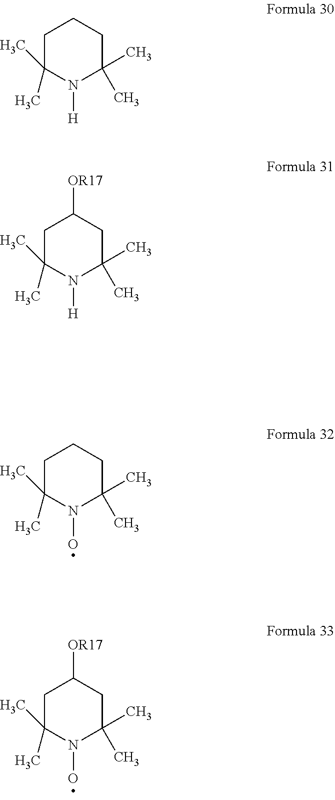
wherein the substituent R17 denotes preferably a hydrogen atom or a benzyl group or a benzoyl group.

12. A device being a smart window or a memory display which consists of a two plastic or glass substrates covered inside with conductive and/or orienting layer, wherein both electrodes are transparent or one contains a reflective layer from a metal preferably aluminum and the gap between substrates is in the range of 5-20 μm, preferably 15 μm, characterized in that said device is filled with a liquid crystalline composition according to claim 4.

13. The method of filling of the device according to claim 12, in which the filling process is carried in an inert atmosphere, preferably in argon for said memory display or in crypton for said smart window and wherein the liquid crystal smectic A composition used is earlier carefully degassed and is stored in said inert atmosphere.

14. A pyrimidine derivative expressed by the general formulae 19 and 20 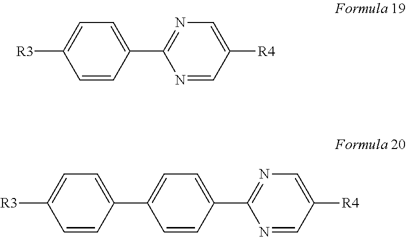
wherein a left terminal substituent R3 is an alkyl carbonato group (H₂ₙ₊₁Cₙ)OCOO and right terminal substituent R4 is an alkyl group CₘH₂ₘ₊₁ or an alcoxy group OCₘH₂ₘ₊₁ or left substituent R3 is an alkyl group H₂ₙ₊₁Cₙ or an alcoxy group H₂ₙ₊₁CnO and right substituent R4 is alkyl carbonato group OCOO CₘH₂ₘ₊₁ in which symbols n and m denote independently numbers from 1 to 15.

15. A smectic A composition according to claim 6, wherein a first kind of ionic dopant is at least one or more ionic dopants with delocalized charge of cation selected from ionic compounds expressed by the general formulae 22-26, 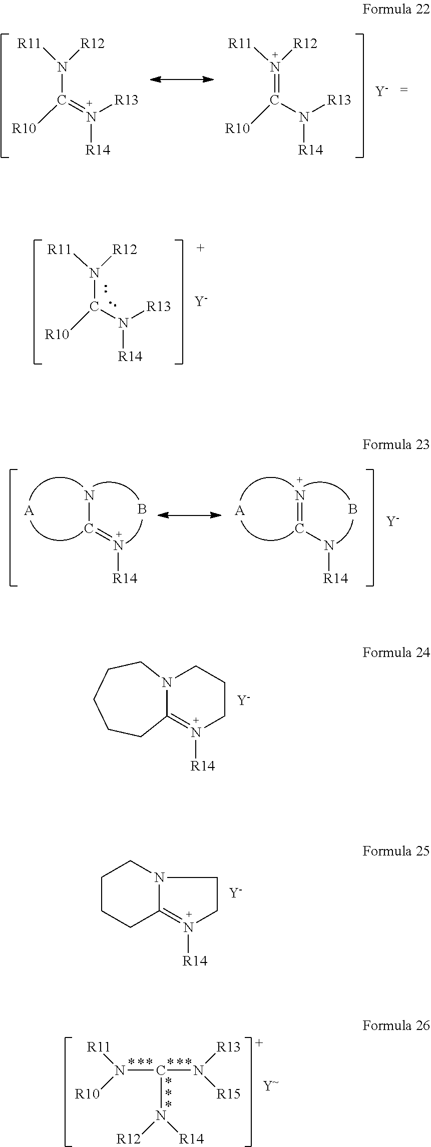
wherein the substituents R10, R11, R13 and R15 in the cationic part of the molecule are independently a hydrogen atom or an alkyl group or an alkylcyclohexyl group or a phenyl group or an alkylphenyl group and they are the same or different and contain 1 to 25 carbon atoms;
the substituents R12 and R14 are an alkyl group containing independently from 1 to 25 carbon atoms, preferably from 12 to 18 carbon atoms;
in the formula 23 symbols A and B denote a part of the ring containing from two to five methylene groups leading to specially preferable compounds with the system of rings expressed by formulae 24 (A=5, B=3) or 25 (A=4, B=2), wherein R14 is preferably an alkyl group containing from 2 to 18 carbon atoms;
the anion Y⁻ denotes an organic or an inorganic anion preferably: Cl^{θ}— (a), Br^{θ}— (b), ClO₄^{θ}— (c), CF₃SO₃^{θ}— (d), R5-SO₃^{θ}— (e), SCN^{θ}— (f),

16. A smectic A composition according to claim 15, wherein a second kind of ionic dopant is at least one or more ionic dopants with a delocalized charge of cation selected from ionic compounds being complexes formed from the salts of alkaline metals preferably from potassium salts and crown ethers preferably expressed by the general formulae 27 and 28, 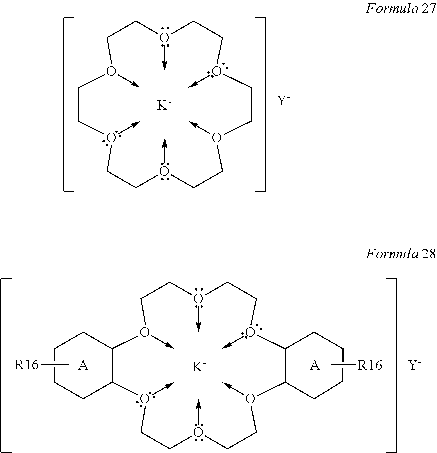
wherein
symbol A is meaning a cyclohexane ring or a benzene ring,
substituents R16 are independently a hydrogen atom or an alkyl group the same or different containing each from 1 to 16 carbon atoms;
anion Y⁻ is an organic anion or an inorganic anion preferably: Cl^{θ}— (a), Br^{θ}— (b), ClO₄^{θ}— (c), CF₃SO₃^{θ}— (d), R5-SO₃^{θ}— (e), SCN^{θ} (f) 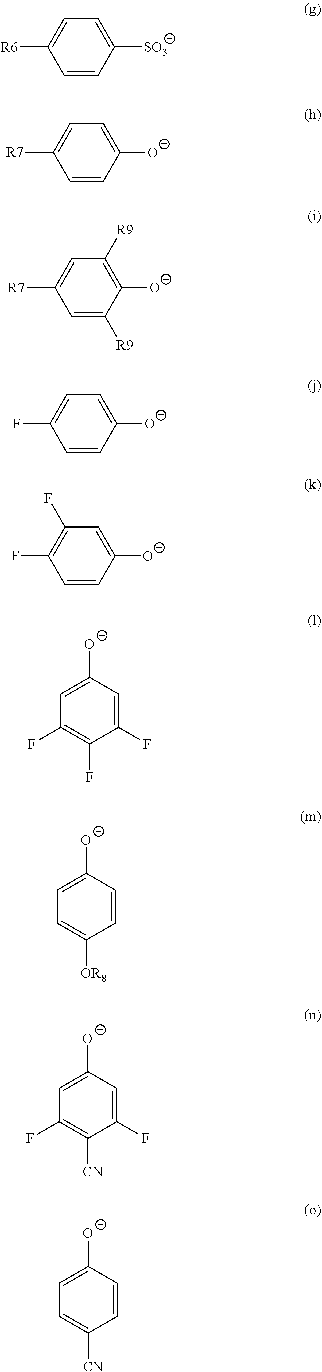 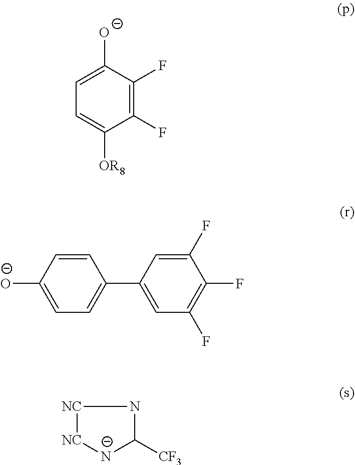
R5, R6, R7 and R8 are meaning a hydrogen or an normal alkyl group or an iso-alkyl group or a sec-alkyl group each containing independently from 1 to 20 carbon atoms and R9 is a branched alkyl group preferably the tert-butyl group.
